(19) United States
(12) Patent Application Publication
(10) Pub. No.: US 2023/0279481 A1
(43) Pub. Date: Sep. 7, 2023

(54) NANO-PARTITIONS FOR MULTIPLE REACTIONS IN PARTITION-BASED ASSAYS

(71) Applicant: 10x Genomics, Inc., Pleasanton, CA (US)

(72) Inventors: Sean Marrache, Concord, CA (US); Shalini Gohil, Castro Valley, CA (US); Joshua Delaney, Northcote (AU); Paul Eugene Lund, San Leandro, CA (US)

(21) Appl. No.: 18/196,835

(22) Filed: May 12, 2023

Related U.S. Application Data
(63) Continuation of application No. PCT/US2021/058527, filed on Nov. 9, 2021.
(60) Provisional application No. 63/113,740, filed on Nov. 13, 2020.

(51) Int. Cl. C12Q 1/6855

(52) U.S. Cl. CPC C12Q 1/6855

(57) ABSTRACT

The present disclosure provides compositions, methods, and kits comprising a nano-partition contained within a partition that allows for the separation and use of at least two enzymes in a partition-based assay of a biological sample, such as a single cell in a droplet. The compositions and methods are useful for carrying out partition-based assays of fixed biological samples.

### FIELD OF THE INVENTION

The present disclosure relates generally to compositions comprising a partition containing a biological sample and at least two enzymes which are separated by a nano-partition, and methods for their use in multi-enzyme partition-based assays.

### BACKGROUND OF THE INVENTION

Biological samples containing a variety of biomolecules can be processed for various purposes, such as detection of a tissue development or disease (e.g., cell differentiation, cancer) or genotyping (e.g., mutation, polymorphism, species identification). Microfluidic technologies have been developed for partitioning individual biological samples (e.g., cells) into discrete partitions. Each discrete partition may be fluidically isolated from other partitions, enabling accurate control of respective environments in the partitions, allowing for each biological sample in a partition to be processed separately. Biological samples in the discrete partitions can be barcoded and subjected to enzymatic, chemical, and/or physical processes such as heating, cooling, or chemical reactions. This allows each discrete partition to contain its own separate assay that can be qualitatively or quantitatively processed.

Biological samples can be unstable. When a biological sample is removed from its viable niche physical decomposition can begin immediately. The degree of decomposition is determined by a number of factors including time, solution buffering conditions, temperature, source (e.g. certain tissues and cells a have higher levels of endogenous RNase activity or dependent on cellular niche), biological stress (e.g. enzymatic tissue dissociation can activate stress response genes or damage cell membranes), and physical manipulation (e.g. pipetting, centrifuging). The degradation includes important nucleic acid molecules (e.g., RNA), proteins, as well as higher-order 3D structure of molecular complexes, whole cells, tissues, organs, and organisms. The instability of biological samples can be a significant obstacle for their use with partition-based assays (e.g., single cell assays). Sample degradation greatly limits the ability to use such assays accurately and reproducibly with a wide range of available biological samples.

The problem of biological sample instability can be mitigated by preserving or fixing the sample using standard biological preservation methods such as cryopreservation, dehydration (e.g., in methanol), high-salt storage (e.g., using RNAssist or RNAlater), and/or chemical fixing agents that create covalent crosslinks (e.g., paraformaldehyde or DSP). The ability to use such a fixed biological sample in a partition-based assay requires that the fixed biological sample can be rapidly and efficiently un-fixed so that the relevant assay can be carried out before sample degradation occurs. Many of the enzymatic reagents used in un-fixing a biological sample, such as proteases which contribute to un-fixing by degrading networks of native proteins in the fixed sample, are incompatible with the enzymatic assay reagents, such as reverse transcriptase, that also must be contained in the partition. Likewise, enzymes for use in other multi-reaction assays such as ligase, transposase, or other polymerases can be similarly incompatible. Thus, compositions and methods are needed that can allow incompatible reagents, such as proteases and nucleic acid processing enzymes (e.g., a reverse transcriptase, a polymerase, a terminal transferase, a transposase, a cas enzyme, a restriction enzyme, a USER enzyme, and/or a ligase), to be contained in a single partition and then used to carry out assays on biological samples.

### SUMMARY OF THE INVENTION

The present disclosure provides compositions and methods comprising a nano-partition contained within a partition that allows for the separation and use of at least two enzymes in a partition-based assay of a biological sample, such as a single cell in a droplet. The nano-partitions are porous such that small molecules (e.g., water, buffer salts, NTPs, inhibitors) but not enzymes, proteins, or other large macromolecules, can pass through them. Thus, the compositions comprising nano-partitions of the present disclosure allow for incompatible enzymes to be used to catalyze reactions within a single partition sequentially (e.g., using a degradable hydrogel nano-partition) or concurrently (e.g., using a non-degradable MOF nano-partition that is porous to substrates and reagents).

In at least one embodiment, the present disclosure provides a composition comprising a partition containing a biological sample, a first enzyme, a second enzyme, and a nano-partition, wherein the nano-partition separates the second enzyme from the first enzyme, thereby preventing the first and second enzymes from interacting. In at least one embodiment, the nano-partition separates the biological sample from the second enzyme. In at least one embodiment, the first and second enzymes are incompatible; optionally, wherein the first enzyme degrades the second enzyme, the first enzyme reduces or reverses the activity of the second enzyme, and/or the first enzyme degrades a substrate or product of a reaction catalyzed by the second enzyme. In at least one embodiment, the nano-partition comprises pores having an average diameter of less than about 5 nm, less than about 4 nm, less than about 3.5 nm, less than about 3 nm, or less than about 2 nm. In at least one embodiment, the nano-partition comprises pores having an average diameter of between about 0.1 nm and about 10 nm, about 0.1 nm and about 5 nm, about 0.1 nm and about 3.5 nm, about 0.1 nm and about 2.5 nm, about 0.1 nm and about 2 nm, about 0.5 nm and about 10 nm, about 1 nm and about 8 nm, about 1.5 nm and about 6 nm, about 2 nm and about 5 nm, or about 2.3 nm and about 4 nm. In at least one embodiment, the nano-partition comprises pores that allow the diffusion of nucleic acids; optionally, the nano-partition comprises pores that allow the diffusion of mRNA molecules.

In at least one embodiment of the composition of the present disclosure, the nano-partition encapsulates the second enzyme.

In at least one embodiment of the composition of the present disclosure, the nano-partition comprises a material selected from a metal organic framework (MOF), a hydrogel matrix, a dendrimersome, or a polymersome; optionally, wherein the nano-partition is a hydrogel matrix comprising cleavable crosslinks. In at least one embodiment, the nano-partition material is a MOF comprising a zeolitic imidazolate framework (ZIF).

In at least one embodiment of the composition of the present disclosure, the nano-partition is degradable; optionally, wherein the nano-pore is degradable by a stimulus selected from heat, UV light, and a chemical reagent.

In at least one embodiment of the composition of the present disclosure, the first enzyme is a protease; optionally, wherein the protease is selected from: alcalase, alkaline proteinase, ArcticZymes Proteinase, bacillopeptidase A, bacillopeptidase B, bioprase, colistinase, esperase, genenase, kazusase, maxatase, pepsin, Serratia peptidase, proteinase K, protease S, savinase, subtilisin A, subtilisin B, subtilisin BL, subtilisin E, subtilisin J, subtilisin S, subtilisin S41, thermoase, and trypsin, or a combination thereof.

In at least one embodiment of the composition of the present disclosure, the second enzyme is a nucleic acid processing enzyme; optionally, wherein the nucleic acid processing enzyme is selected from: a reverse transcriptase (RT), a polymerase, a terminal transferase, a transposase, a cas enzyme, a restriction enzyme, a USER enzyme, and/or a ligase.

In at least one embodiment of the composition of the present disclosure, the partition is a discrete droplet.

In at least one embodiment of the composition of the present disclosure, the partition further comprises a barcode; optionally, wherein the barcode comprises a bead.

In at least one embodiment of the composition of the present disclosure, the partition further comprises assay reagents; optionally, wherein the assay reagents comprise cDNA synthesis reagents; optionally, wherein the cDNA synthesis reagents comprise NTPs, primers, and template switch oligonucleotides.

In at least one embodiment of the composition of the present disclosure, the biological sample is derived from a tissue sample, a biopsy sample, or a blood sample; optionally, wherein the biological sample is a single cell, an organelle of a single cell, and/or a nuclei of a single cell.

In at least one embodiment of the composition of the present disclosure, the biological sample is a fixed biological sample. In at least one embodiment, the partition further comprises an un-fixing agent; optionally, wherein the un-fixing agent comprises a compound selected from 2-amino-5-methylbenzoic acid (compound (1)), 2-amino-5-nitrobenzoic acid (compound (2)), (2-amino-5-methylphenyl)phosphonic acid (compound (3)), 2-amino-5-methylbenzenesulfonic acid (compound (4)), 2,5-diaminobenzenesulfonic acid (compound (5)), 2-amino-3,5-dimethylbenzenesulfonic acid (compound (6)), (2-amino-5-nitrophenyl)phosphonic acid (compound (7)), (4-aminopyridin-3-yl)phosphonic acid (compound (8)), (3-aminopyridin-2-yl)phosphonic acid (compound (9)), (5-aminopyrimidin-4-yl)phosphonic acid (compound (10)), (2-amino-5-{[2-(2-poly-ethoxy)ethyl]carbamoyl}phenyl)phosphonic acid (compound (11)), or a combination thereof.

The present disclosure also provides methods for preparing a sample. In at least one embodiment, the method of preparing a sample comprises: generating a partition containing a biological sample, a first enzyme, a second composition enzyme, and a nano-partition, wherein the nano-partition separates the second enzyme from the first enzyme, thereby preventing the first and second enzymes from interacting. In at least one embodiment, the nano-partition comprises pores having an average diameter of less than about 5 nm, less than about 4 nm, less than about 3.5 nm, less than about 3 nm, or less than about 2 nm. In at least one embodiment, the nano-partition comprises pores having an average diameter of between about 0.1 nm and about 10 nm, about 0.1 nm and about 5 nm, about 0.1 nm and about 3.5 nm, about 0.1 nm and about 2.5 nm, about 0.1 nm and about 2 nm, about 0.5 nm and about 10 nm, about 1 nm and about 8 nm, about 1.5 nm and about 6 nm, about 2 nm and about 5 nm, or about 2.3 nm and about 4 nm. In at least one embodiment, the nano-partition comprises pores that allow the diffusion of nucleic acids; optionally, wherein the nano-partition comprises pores that allow the diffusion of mRNA molecules.

In at least one embodiment of the method, the nano-partition encapsulates the second enzyme.

In at least one embodiment of the method, the nano-partition comprises a material selected from a metal organic framework, a hydrogel matrix, a dendrimersome, or a polymersome; optionally, wherein the nano-partition is a hydrogel matrix comprising cleavable crosslinks. In at least one embodiment, the nano-partition material is a MOF comprising a zeolitic imidazolate framework (ZIF).

In at least one embodiment of the method, the first enzyme is a protease; optionally, wherein the protease is selected from: alcalase, alkaline proteinase, ArcticZymes Proteinase, bacillopeptidase A, bacillopeptidase B, bioprase, colistinase, esperase, genenase, kazusase, maxatase, pepsin, Serratia peptidase, proteinase K, protease S, savinase, subtilisin A, subtilisin B, subtilisin BL, subtilisin E, subtilisin J, subtilisin S, subtilisin S41, thermoase, and trypsin, or a combination thereof.

In at least one embodiment of the method, the second enzyme is a nucleic acid processing enzyme; optionally, wherein the nucleic acid processing enzyme is selected from: a reverse transcriptase, a polymerase, a terminal transferase, a cas enzyme, a restriction enzyme, a USER enzyme, a transposase, and/or a ligase.

In at least one embodiment, the nano-partition is degradable; optionally, wherein the nano-pore is degradable by a stimulus selected from heat, UV light, and a chemical reagent.

In at least one embodiment of the method, the partition is a discrete droplet.

In at least one embodiment of the method, the partition further comprises a barcode; optionally, wherein the barcode comprises a bead.

In at least one embodiment of the method, the partition further comprises assay reagents. In at least one embodiment, the assay reagents comprise cDNA synthesis reagents; optionally, wherein the cDNA synthesis reagents comprise NTPs, primers, and template switch oligonucleotides.

In at least one embodiment of the method, the biological sample is derived from a tissue sample, a biopsy sample, or a blood sample; optionally, wherein the biological sample is a single cell, an organelle of a single cell, and/or nuclei of a single cell.

In at least one embodiment of the method, the biological sample is a fixed biological sample.

In at least one embodiment of the method, wherein the partition further comprises an un-fixing agent; optionally, wherein the un-fixing agent is a composition comprising a compound selected from compound (1), compound (2), compound (3), compound (4), compound (5), compound (6), compound (7), compound (8), compound (9), compound (10), compound (11), or a combination thereof.

The present disclosure also provides assay methods. In at least one embodiment, the assay method comprises: (a) providing a partition containing a biological sample, a first enzyme, a second enzyme, and a nano-partition, wherein the first and second enzymes catalyze different reactions, and wherein the nano-partition separates the second enzyme from the biological sample and the first enzyme, thereby preventing the first and second enzymes from interacting; (b) wherein the first enzyme catalyzes a reaction with the biological sample; and (c) wherein the second enzyme catalyzes a reaction with the biological sample, or a component thereof. In at least one embodiment, steps (b) and (c) occur simultaneously. In at least one embodiment, step (c) occurs after (b).

In at least one embodiment of the assay method, the first and second enzymes are incompatible; optionally, wherein the first enzyme degrades the second enzyme, and/or reduces the activity of the second enzyme.

In at least one embodiment, the reaction of the first enzyme with the biological sample generates a substrate for the reaction of the second enzyme; optionally wherein the generating comprises rendering the substrate accessible to the second enzyme.

In at least one embodiment of the assay method, the reaction of the second enzyme with the biological sample generates analytes. In at least one embodiment, the assay method further comprises a step of detecting the generated analytes.

In at least one embodiment, the nano-partition comprises pores having an average diameter of less than about 5 nm, less than about 4 nm, less than about 3.5 nm, less than about 3 nm, or less than about 2 nm. In at least one embodiment, the nano-partition comprises pores having an average diameter of between about 0.1 nm and about 10 nm, about 0.1 nm and about 5 nm, about 0.1 nm and about 3.5 nm, about 0.1 nm and about 2.5 nm, about 0.1 nm and about 2 nm, about 0.5 nm and about 10 nm, about 1 nm and about 8 nm, about 1.5 nm and about 6 nm, about 2 nm and about 5 nm, or about 2.3 nm and about 4 nm. In at least one embodiment, the nano-partition comprises pores that allow the diffusion of nucleic acids; optionally, wherein the nano-partition pores allow the diffusion of mRNA molecules.

In at least one embodiment of the assay method, the nano-partition encapsulates the second enzyme.

In at least one embodiment of the assay method, the nano-partition comprises a material selected from a metal organic framework, a hydrogel matrix, a dendrimersome, or a polymersome; optionally, wherein the nano-partition is a hydrogel matrix comprising cleavable crosslinks. In at least one embodiment, the nano-partition material is a MOF comprising a zeolitic imidazolate framework (ZIF).

In at least one embodiment of the assay method, the first enzyme is a protease; optionally, wherein the protease is selected from: alcalase, alkaline proteinase, ArcticZymes Proteinase, bacillopeptidase A, bacillopeptidase B, bioprase, colistinase, esperase, genenase, kazusase, maxatase, pepsin, Serratia peptidase, proteinase K, protease S, savinase, subtilisin A, subtilisin B, subtilisin BL, subtilisin E, subtilisin J, subtilisin S, subtilisin S41, thermoase, and trypsin, or a combination thereof..

In at least one embodiment of the assay method, the second enzyme is a nucleic acid processing enzyme; optionally, wherein the nucleic acid processing enzyme is selected from: a reverse transcriptase, a polymerase, a terminal transferase, a cas enzyme, a restriction enzyme, a USER enzyme, a transposase, and/or a ligase.

In at least one embodiment of the assay method, the partition is a discrete droplet.

In at least one embodiment of the assay method, the partition further comprises a barcode; optionally, wherein the barcode comprises a bead.

In at least one embodiment of the assay method, the partition further contains assay reagents. In at least one embodiment, the assay reagents comprise cDNA synthesis reagents; optionally, wherein the cDNA synthesis reagents comprise NTPs, primers, and template switch oligonucleotides.

In at least one embodiment of the assay method, the biological sample is derived from a tissue sample, a biopsy sample, or a blood sample; optionally, wherein the biological sample is a single cell, an organelle of a single cell, and/or a nucleus of a single cell.

In at least one embodiment of the assay method, the biological sample is a fixed biological sample.

In at least one embodiment of the assay method, the partition further comprises an unfixing agent; optionally, wherein the un-fixing agent is a composition comprising a compound selected from compound (1), compound (2), compound (3), compound (4), compound (5), compound (6), compound (7), compound (8), compound (9), compound (10), compound (11), or a combination thereof.

In at least one embodiment of the assay method, the nano-partition is degradable and the method further comprises a step of providing a stimulus that degrades the nano-partition; optionally, wherein the nano-partition is degradable by a stimulus selected from heat, UV light, and a chemical reagent. In at least one embodiment, the stimulus that degrades the nano-partition also deactivates the first enzyme. In at least one embodiment, the stimulus is a chemical reagent; optionally, wherein the chemical reagent is selected from DTT, DETA, EDA, TETA, hydrazine monohydrate, or a combination thereof.

In at least one embodiment of the assay method, the method further comprises a step of deactivating the first enzyme.

In at least one embodiment of the assay method, the partition is a discrete droplet and the method further comprises emulsifying the droplet, thereby releasing its contents.

The present disclosure also provides kits useful for carrying out the methods. In at least one embodiment, the present disclosure provides a kit comprising: assay reagents; a first enzyme; and a second enzyme encapsulated in a nano-partition.

In at least one embodiment of the kit, the nano-partition comprises pores having an average diameter of less than about 5 nm, less than about 4 nm, less than about 3.5 nm, less than about 3 nm, or less than about 2 nm. In at least one embodiment, the nano-partition comprises pores having an average diameter of between about 0.1 nm and about 10 nm, about 0.1 nm and about 5 nm, about 0.1 nm and about 3.5 nm, about 0.1 nm and about 2.5 nm, about 0.1 nm and about 2 nm, about 0.5 nm and about 10 nm, about 1 nm and about 8 nm, about 1.5 nm and about 6 nm, about 2 nm and about 5 nm, or about 2.3 nm and about 4 nm. In at least one embodiment, the nano-partition comprises pores that allow the diffusion of mRNA molecules.

In at least one embodiment of the kit, the nano-partition is degradable; optionally, wherein the nano-pore is degradable by a stimulus selected from heat, UV light, and a chemical reagent.

In at least one embodiment, the kit further comprises a barcode; optionally, wherein the barcode comprises a bead.

In at least one embodiment of the kit, the first enzyme is a protease; optionally, wherein the protease is selected from: alcalase, alkaline proteinase, ArcticZymes Proteinase, bacillopeptidase A, bacillopeptidase B, bioprase, colistinase, esperase, genenase, kazusase, maxatase, pepsin, Serratia peptidase, proteinase K, protease S, savinase, subtilisin A, subtilisin B, subtilisin BL, subtilisin E, subtilisin J, subtilisin S, subtilisin S41, thermoase, and trypsin, or a combination thereof.

In at least one embodiment, the kit further comprises an un-fixing agent; optionally, wherein the un-fixing agent is a composition comprising a compound selected from compound (1), compound (2), compound (3), compound (4), compound (5), compound (6), compound (7), compound (8), compound (9), compound (10), compound (11), or a combination thereof.

In at least one embodiment of the kit, the second enzyme is a nucleic acid processing enzyme; optionally, wherein the nucleic acid processing enzyme is selected from: a reverse transcriptase, a polymerase, a terminal transferase, a cas enzyme, a restriction enzyme, a USER enzyme, a transposase, and/or a ligase.

In at least one embodiment of the kit, the assay reagents comprise cDNA synthesis reagents; optionally, wherein the cDNA synthesis reagents comprise NTPs, primers, and template switch oligonucleotides.

In at least one embodiment of the kit, the nano-partition comprises a material selected from a metal organic framework, a hydrogel matrix, a dendrimersome, or a polymersome. In at least one embodiment, the nano-partition is a hydrogel matrix comprising cleavable crosslinks. In at least one embodiment, the nano-partition material is a MOF comprising a zeolitic imidazolate framework (ZIF).

### BRIEF DESCRIPTION OF THE FIGURES

A better understanding of the novel features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 shows an example of a microfluidic channel structure for partitioning individual cells or particles of a biological sample.

FIG. 2 shows an example of a microfluidic channel structure for delivering barcode carrying beads to droplets.

FIG. 3 shows an example of a microfluidic channel structure for co-partitioning a biological samples and reagents.

FIG. 4 shows an example of a microfluidic channel structure for the controlled partitioning of beads into discrete droplets.

FIG. 5 shows an example of a microfluidic channel structure for increased droplet generation throughput.

FIG. 6 shows another example of a microfluidic channel structure for increased droplet generation throughput.

FIG. 7 shows an exemplary barcode carrying bead.

FIG. 8 shows another exemplary barcode carrying bead.

FIG. 9 shows an exemplary microwell array schematic.

FIG. 10 shows an exemplary microwell array workflow for processing nucleic acid molecules.

FIG. 11 schematically illustrates examples of labelling agents.

FIG. 12 depicts an example of a barcode carrying bead.

FIG. 13A, FIG. 13B, and FIG. 13C schematically depict an example workflow for processing nucleic acid molecules.

FIG. 14 depicts a schematic illustrating the formation of an exemplary embodiment of a nano-partition containing the enzyme, reverse transcriptase (RT).

FIG. 15A and FIG. 15B depict a schematic illustration of an exemplary embodiment of a partition-based assay method that includes a fixed biological sample, a protease, an un-fixing agent, and a degradable nano-partition that separates a reverse transcriptase (RT), that it is incompatible with the protease, within the partition.

### DETAILED DESCRIPTION

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of these limits, ranges excluding (i) either or (ii) both of those included limits are also included in the invention. For example, “1 to 50,” includes “2 to 25,” “5 to 20,” “25 to 50,” “1 to 10,” etc.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

Generally, the nomenclature used herein and the techniques and procedures described herein include those that are well understood and commonly employed by those of ordinary skill in the art, such as the common techniques and methodologies described in e.g., Green and Sambrook, Molecular Cloning: A Laboratory Manual (Fourth Edition), Vols. 1-3, Cold Spring Harbor Laboratory, Cold Spring Harbor, N.Y., 2012 (hereinafter “Sambrook”); and Current Protocols in Molecular Biology, F. M. Ausubel et al., eds., originally published in 1987 in book form by Greene Publishing Associates, Inc. and John Wiley & Sons, Inc., and regularly supplemented through 2011, and now available in journal format online as Current Protocols in Molecular Biology, Vols. 00 - 130, (1987-2020), published by Wiley & Sons, Inc. in the Wiley Online Library (hereinafter “Ausubel”).

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present invention pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

### A. Nano-Partitions for Separating Enzymes Within a Partition

The ability to generate discrete partitions, such as aqueous droplets, that contain distinct biological samples, such as single cells, has led to the development of a range of partition-based assays. See e.g., US Pat. Nos. 9,694,361, 10,357,771, 10,273,541, and 10,011,872, and US Pat. Publ. Nos. 2018/0105808A1, 2019/0367982A1, and 2019/0338353A1, each of which is hereby incorporated herein by reference in its entirety. The use of partitions to carry out assays involving contacting a partitioned biological sample with a sequence of two or more enzymes has remained difficult or impossible where interactions between the two or more enzymes can interfere with the accuracy and/or sensitivity of an assay. In particular, partition-based assays involving the use of a protease, e.g., to initially lyse a biological sample (e.g., a tissue sample, a biopsy sample, a blood sample, a single cell, an organelle of a single cell, and/or a nucleus of a single cell), are challenging because the protease are relatively nonspecific and will inactivate the other enzymes required for the assay (e.g., reverse transcriptase). Attempts to inactivate a first enzyme (e.g., by heat or inhibition) in a partition after it has carried out its desired enzymatic task (e.g., sample lysis) but before it interacts deleteriously with a second enzyme in the partition which is needed at later stage of the assay have had little success due to the sensitivity of partition-based biochemistry. The problem has remained how to maintain some separation within the partition between the first and second enzyme. The present disclosure solves the problem of using two (or more) incompatible enzymes in a single partition through the use of a nano-partition contained within the partition to separate the enzymes. The pores of the nano-partition have an average diameter of between about 0.1 nm and about 10 nm, and/or allow the diffusion of nucleic acids, such as mRNA molecules. Accordingly, in at least one embodiment, the present disclosure provides a composition comprising a partition containing a biological sample, a first enzyme, a second enzyme, and a nano-partition, wherein the nano-partition separates the second enzyme from the biological sample and the first enzyme, thereby preventing the first and second enzymes from interacting but also allowing nucleic acids (and other reagents) to diffuse through the nano-partition and thereby interact with both enzymes For example, in the case of the first enzyme being a protease and the second enzyme being a reverse transcriptase (RT), the mRNA molecules released by the protease treatment of a biological sample in the partition are able to undergo catalysis by the RT which has been protected from the proteolytic activity of the protease by the nano-partition.

“Biological sample,” as used herein refers to any sample of biological origin that includes a biomolecule, such as a nucleic acid, a protein, a carbohydrate, and/or a lipid. Biological samples used in the methods and compositions of the disclosure include blood and other liquid samples of biological origin, solid tissue samples such as a tissue sample (i.e., tissue specimen), a biopsy (i.e., a biopsy specimen), a tissue culture, cells derived therefrom, the progeny of derived cells, single cells, and organelles derived from single cells, including nuclei. This includes samples that have been manipulated in any way after isolation from the biological source, such as by treatment with reagents (e.g., fixation reagents, thereby generating a fixed biological sample); samples such as tissues that are embedded in medium (e.g., paraffin); sectioned tissue sample (e.g., sectioned samples that are mounted on a solid substrate such as a glass slide); washed; or enrichment for certain cell populations, such as cancer cells, neurons, stem cells, etc. The term also encompasses samples that have been enriched for particular types of molecules, e.g., nucleic acids, polypeptides, etc. The term also encompasses a clinical sample, and also includes tissue obtained by surgical resection, tissue obtained by biopsy, cells in culture, cell supernatants, cell lysates, tissue samples (i.e., tissue specimens), organs, bone marrow, blood, plasma, serum, and the like. Biological sample also is intended to include a sample obtained from a patient’s cancer cell, e.g., a sample comprising polynucleotides and/or polypeptides that is obtained from a patient’s cancer cell (e.g., a cell lysate or other cell extract comprising polynucleotides and/or polypeptides); and a sample having cells (e.g., cancer cells) from a patient.

It is contemplated that the biological samples used in the compositions and methods of the present disclosure can be derived from another sample. Biological samples can include a tissue sample, such as a biopsy, core biopsy, needle aspirate, or fine needle aspirate. Biological samples also include a biological fluid sample, such as a blood sample, urine sample, or saliva sample, or the biological sample may be a skin sample, a cheek swab. The biological sample may be a plasma or serum sample. The biological sample may include cells or be a cell-free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

“Partition,” as used herein, refers to a space or volume that is suitable to contain one or more species or conduct one or more reactions. A partition may be a physical compartment, such as a droplet or well (e.g., a microwell). The partition may isolate the space or volume from another space or volume. The partition may be a “discrete droplet” of a first phase (e.g., aqueous phase) in a second phase (e.g., oil) that is immiscible with the first phase. In at least one embodiment, the partition used in the compositions and methods of the present is a discrete droplet in an immiscible phase. Methods and materials for generating biological samples in such discrete droplets are known in the art and described in greater detail elsewhere herein.

“Droplet-based assay” refers to an assay carried out on a biological sample contained within a discrete droplet in an emulsion. The discrete droplet usually also includes a unique identifier for the sample in the form of a unique oligonucleotide sequence also contained in the discrete droplet. The discrete droplet can also contain the assay reagents that are used to generate detectable analytes (e.g., 3′ cDNA sequences) from the sample and provide useful information about it (e.g., RNA transcript profile). Further details of methods and compositions for carrying out droplet-based assays are provided elsewhere herein.

“Nano-partition” as used herein refers to a material or particle that retain or contain an enzyme molecule within a sub-volume of a partition. Nano-partitions of the present disclosure have dimensions on the order of about 3 nm (or 0.003 µm) to about 10 µm (or 10,000 nm) and are made of porous materials that allow small molecules (e.g., water, buffer salts, NTPs, inhibitors) but not enzymes, proteins, or other large macromolecules, to pass through. Accordingly, the pores of the nano-partition have an average diameter typically in the range of about 0.1 nm and about 10 nm, and in some embodiments, an average diameter of less than about 5 nm. As described elsewhere herein, the nano-partition comprises pores that can have an average diameter of between about 0.1 nm and about 5 nm, about 0.1 nm and about 3.5 nm, about 0.1 nm and about 2.5 nm, about 0.1 nm and about 2 nm, about 0.5 nm and about 10 nm, about 1 nm and about 8 nm, about 1.5 nm and about 6 nm, about 2 nm and about 5 nm, or about 2.3 nm and about 4 nm. A range of exemplary porous materials useful for nano-partitions are contemplated herein and can include a metal organic framework (MOF), a hydrogel matrix, a dendrimersome, or a polymersome. Such materials and methods for their preparation are known in the art and described elsewhere herein.

In at least one embodiment, the nano-partition comprises a zeolitic imidazolate framework (ZIF) metal organic framework (MOF). The preparation of a ZIF containing an enzyme, such as catalase, which is protects the enzyme from protease digestion in solution has been described by e.g., Shieh, et al., “Imparting Functionality to Biocatalysts via Embedding Enzymes into Nanoporous Materials by a de Novo Approach: Size-Selective Sheltering of Catalase in Metal-Organic Framework Microcrystals,” J Am Chem Soc.137(13):4276-9 (2015). For example, the ZIF-90 described by Shieh et al., is synthesized in the presence of catalase and results in the enzyme embedded within 2 µm ZIF-90 crystals. The ZIF-90 crystals are a porous MOF that allows the enzyme to act on its peroxide substrate in solution, while in the presence of proteinase K, which otherwise would rapid proteolyze the catalase and destroy its activity. Catalase encapsulated in ZIF-90 thus demonstrates that a MOF can act as a nano-partition capable of enzyme containment and protection from incompatible enzymes in the same solution. A variety of alternatively sized ZIF MOFs capable of containing enzymes are further referenced in the Shieh et al. (2015) reference.

In another relevant example, the water-stable mesoporous channel-type zirconium MOF, PCN-128y, has been used to encapsulate the enzyme, organophosphorus acid anhydrolase (OPAA) in a nano-partition in which the enzyme retains its activity even under high heat. See, Li et al., “Encapsulation of a Nerve Agent Detoxifying Enzyme by a Mesoporous Zirconium Metal-Organic Framework Engenders Thermal and Long-Term Stability,” J. Am. Chem. Soc.2016, 138, 26, 8052-8055. The OPAA enzyme encapsulation in the MOF can be performed rapidly under mild conditions and requires no prior chemical modification of the enzyme.

“Degradable nano-partition” as used herein refers to a nano-partition that can be degraded, dissolved, or disrupted by a chemical and/or physical stimulus thereby allowing any molecules being retained or contained by the nano-partition to be released and diffuse into the larger partition.

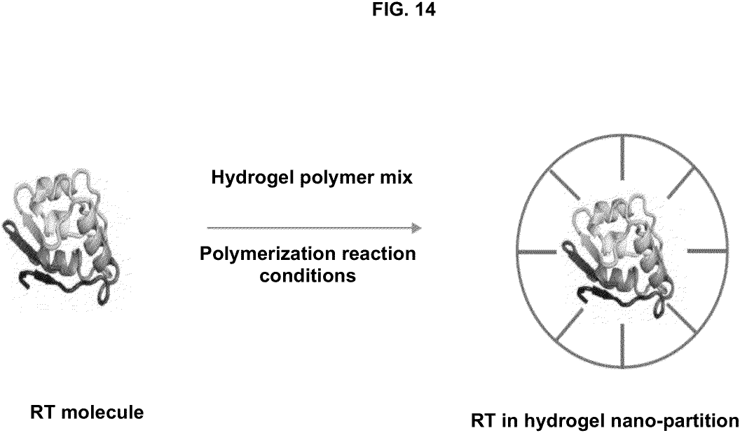

In at least one embodiment, the degradable nano-partition is a particle that encapsulates a macromolecule (e.g., an enzyme) within a degradable material. For example, the degradable nano-partition can be a hydrogel particle with an enzyme molecule embedded within the hydrogel matrix of degradable (e.g., cleavable) crosslinks, such as disulfide or carbamate linkages. FIG. 14 provides a schematic depiction of the formation of an exemplary embodiment of a reverse transcriptase enzyme contained in a nano-partition made of a hydrogel matrix. The nano-partition forms when hydrogel matrix polymerizes (e.g., undergoes gelation) and entraps the RT molecule thereby preventing other macromolecules in a solution that are larger than the hydrogels average pore size (such as an incompatible protease) from interacting with the RT molecule. Degradation of the hydrogel matrix forming the nano-partition, e.g., by heat and/or treatment with DTT that cleaves disulfide crosslinks, can be used to release the RT into solution, e.g., after the incompatible protease has been inhibited, denatured, and/or otherwise neutralized. Materials and methods for preparing a degradable hydrogel matrix with an embedded or entrapped cells and/or macromolecules, such as an enzyme, and techniques for degrading the hydrogels are known in the art, and described in e.g., U.S. Pat. Publ. Nos. 2019/0100632A1, and 2019/0233878A1, each of which is hereby incorporated by reference herein), and described in greater detail elsewhere herein.

In at least one embodiment, degradation of the hydrogel matrix can be carried out by cleaving the crosslinks that form the matrix, the timing of the crosslink cleaving reaction can be controlled in time using a chemical or physical stimulus to carry out the reaction. In at least one embodiment, the stimulus that degrades the nano-partition (e.g., by cleaving the hydrogel crosslinks) is selected from heat, UV light, and a chemical reagent. For example, the hydrogel matrix degradation can be initiated by the physical stimulus of heat applied to the partition, wherein the heat simultaneously inactivates the incompatible enzyme (e.g., a protease) in the larger partition. Accordingly, in at least one embodiment, the stimulus that degrades the nano-partition, also deactivates the incompatible first enzyme that is present outside the nano-partiton (e.g., in the larger partition), thereby minimizing or preventing any deleterious interaction between the two enzymes.

The compound, dithiothreitol (“DTT”) is a chemical reagent that is well known for its ability to reductively cleave disulfide bonds. The compound diethylenetriamine (“DETA”) is effective for the cleavage of carbamate bonds. See e.g., Noshita et al., “Diethylenetriamine-Mediated Direct Cleavage of Unactivated Carbamates and Ureas,” Org. Lett. 18: 6062-6065 (2016). Similarly, the diamine compounds, including ethylenediamine (“EDA”), triethylenetetramine (“TETA”), and hydrazine monohydrate can cleave carbamate bonds. Accordingly, in at least one embodiment wherein the degradable nano-partition comprises disulfide and/or carbamate crosslinks, the stimulus used to degrade the nano-partion can be a chemical reagent; optionally, wherein the chemical reagent is selected from DTT, DETA, EDA, TETA, hydrazine monohydrate, or a combination thereof. In such an embodiment, the incompatible first enzyme in the larger outer partition can be inactivated via a chemical (e.g., inhibitor) or physical stimulus (e.g., heat), and then the hydrogel matrix crosslinks of the nano-partition can be cleaved using chemical agent (e.g., DTT), thereby releasing the enzyme from the nano-partition into the larger partition after the incompatible enzyme has been inactivated.

As is described above, it is contemplated that in the partition-based assay method embodiments of the present disclosure, the method further comprises a step of inactivating a first enzyme in the larger outer partition before degrading the nano-partition to release the second enzyme into the outer partition with the biological sample. In at least one embodiment of the assay methods of the present disclosure, the first enzyme is a protease which is useful for acting proteolytically on the biological sample e.g., to release biological material such as RNA from a cell. However, the protease activity can also destroy the activity of other enzymes contained in the partition as reagents in the assay method (e.g., reverse transcriptase for synthesizing cDNA). For example, in at least one embodiment, the first enzyme in the partition is a protease selected from alcalase, alkaline proteinase, ArcticZymes Proteinase, bacillopeptidase A, bacillopeptidase B, bioprase, colistinase, esperase, genenase, kazusase, maxatase, pepsin, Serratia peptidase, proteinase K, protease S, savinase, subtilisin A, subtilisin B, subtilisin BL, subtilisin E, subtilisin J, subtilisin S, subtilisin S41, thermoase, and trypsin, or a combination thereof; and the second enzyme is selected from: a reverse transcriptase, a polymerase, a terminal transferase, a cas enzyme, a restriction enzyme, a USER enzyme, a transposase, and/or a ligase.

The ability to include a protease as first enzyme in a partition along with a second enzyme in a nano-partition is particularly useful when the sample is a fixed biological sample. Accordingly, in at least one embodiment of the assay methods of the present disclosure, the biological sample is a fixed biological sample. As described in greater detail elsewhere herein, the ability to carry out partition-based assay methods, such as single-cell RNA expression analysis, on fixed biological samples greatly facilitates sample preparation because it prevents the rapid deterioration that fresh biological samples often undergo before the complete assay can be run. Typically, in using fixed biological samples (e.g., PFA-fixed cells or tissue), the preparation of the sample in the partition includes not only a protease but also includes an un-fixing agent. Unfixing agents useful in the methods of the present disclosure can include small molecules that act catalytically in cleaving the aminal and hemi-aminal crosslinks between RNA and DNA that form upon the use of aldehyde fixing agent, such as PFA. Accordingly, in at least one embodiment of the compositions and methods of the present disclosure, the partition further comprises an un-fixing agent. A range of un-fixing agents (or de-crosslinking agents) are available and described in greater detail elsewhere herein. In at least one embodiment, the un-fixing agent is a compound selected from any one of the following catalytic un-fixing agent compounds described elsewhere herein: compound (1), compound (2), compound (3), compound (4), compound (5), compound (6), compound (7), compound (8), compound (9), compound (10), compound (11), or a combination thereof.

As described elsewhere herein, it is contemplated that the partition-based assay methods comprising a nano-partition separating a first enzyme and a second enzyme can further comprise additional assay reagents and barcoding materials. Accordingly, in at least one embodiment of the compositions and methods, the partition further contains assay reagents such as cDNA synthesis reagents. For example, the cDNA synthesis reagents can include nucleotide triphosphate (NTP) substrates for synthesis of cDNA, primers, and template switch oligonucleotides. Additionally, in at least one embodiment, the partition further contains a barcode molecule that can allows for identification of the biological sample in the partition, typically after an assay is carried out and the resulting analytes have been released from the partition and detected (e.g., by sequencing). In at least one embodiment of the assay method, the partition is a discrete droplet and the method further comprises emulsifying the droplet, thereby releasing its contents. As described elsewhere herein, the use of barcodes comprising oligonucleotides in partition-based assays is known in the art. In at least one embodiment, the barcode molecule (e.g., an oligonucleotide) contained in the partition comprises a bead.

### B. Use of Nano-Partitions in Partition-Based Assays With Fixed Biological Samples

Preparation of a biological sample that is useful in a partition-based assay involves numerous steps (e.g., sample transport, tissue dissociation, liquid phase washing and transfer, library preparation). These steps can take from a few hours to days. During this preparation time an un-fixed biological sample will begin to degrade, and decompose resulting in significant loss of sample quality and potentially leading to assay results that do not reflect the natural state of the sample. The ability to use a fixed biological sample in a partition-based assay can be very advantageous.

The ability to use a fixed biological sample in a partition-based assay (e.g., a single cell assay), however, requires rapid and efficient un-fixing of the sample to obtain access to the relevant cellular analytes for processing and/or detection before degradation occurs. Ideally, the partition-based assay data obtained from an un-fixed biological sample should be identical to that obtained from a partition-based assay of a fresh sample, or resemble a sample obtained from its natural environment as closely as possible. Materials and methods for carrying out partition assays using a fixed biological sample, along with protease treatment, and un-fixing agents is described in U.S. Provisional Appl. Nos. 62/952,670, filed Dec. 23, 2019, 62/952,677, filed Dec. 23, 2019, 63/026,500, filed May 18, 2020, and 63/026,513, filed May 18, 2020, each of which is hereby incorporated by reference herein.

Fixation of cell or cellular constituent, or a tissue comprising a plurality of cells or nuclei, may comprise application of a chemical species or chemical stimulus. The term “fixed” as used herein with regard to biological samples refers the state of being preserved from decay and/or degradation. “Fixation” refers to a process that results in a fixed sample, and can include contacting the biomolecules within a biological sample with a fixative (or fixation reagent) for some amount of time, whereby the fixative results in covalent bonding interactions such as crosslinks between biomolecules in the sample. A “fixed biological sample” refers to a biological sample that has been contacted with a fixation reagent or fixative. For example, a formaldehyde-fixed biological sample has been contacted with the fixation reagent formaldehyde. “Fixed cells” or “fixed tissues” refer to cells or tissues that have been in contact with a fixative under conditions sufficient to allow or result in the formation of intra- and inter-molecular covalent crosslinks between biomolecules in the biological sample.

Generally, contact of biological sample (e.g., a cell or nucleus) with a fixation reagent (e.g., paraformaldehyde or PFA) results in the formation of intra- and inter-molecular covalent crosslinks between biomolecules in the biological sample. In some cases, provision of the fixation reagent, such as formaldehyde, may result in covalent aminal crosslinks within RNA, DNA, and/or protein molecules. For example, the widely used fixative reagent, paraformaldehyde or PFA, fixes tissue samples by catalyzing crosslink formation between basic amino acids in proteins, such as lysine and glutamine. Both intra-molecular and inter-molecular crosslinks can form in the protein. These crosslinks can preserve protein secondary structure and also eliminate enzymatic activity in the preserved tissue sample. Examples of fixation reagents include but are not limited to aldehyde fixatives (e.g., formaldehyde, also commonly referred to as “paraformaldehyde,” “PFA,” and “formalin”; glutaraldehyde; etc.), imidoesters, NHS (N-Hydroxysuccinimide) esters, and the like. Other examples of fixation reagents include, for example, organic solvents such as alcohols (e.g., methanol or ethanol), ketones (e.g., acetone), and aldehydes (e.g., paraformaldehyde, formaldehyde (e.g., formalin), or glutaraldehyde). As described herein, cross-linking agents may also be used for fixation including, without limitation, disuccinimidyl suberate (DSS), dimethylsuberimidate (DMS), formalin, and dimethyladipimidate (DMA), dithio-bis(-succinimidyl propionate) (DSP), disuccinimidyl tartrate (DST), and ethylene glycol bis(succinimidyl succinate) (EGS). In some cases, a cross-linking agent may be a cleavable cross-linking agent (e.g., thermally cleavable, photocleavable, etc.). In some cases, more than one fixation reagent can be used in combination when preparing a fixed biological sample.

Changes to a characteristic or a set of characteristics of a cell or cellular constituents (e.g., incurred upon interaction with one or more fixation agents) may be at least partially reversible (e.g., via rehydration, and/or de-crosslinking or un-fixing). “Un-fixed” refers to the processed condition of a biological sample, such as a cell, a plurality of cells, a tissue sample or any other biological sample that is characterized by a prior state of fixation followed by a reversal of the prior state of fixation. As used herein, an un-fixed cell may also be referred to as a “previously fixed” cell. Typically, an un-fixed biological sample is characterized by broken or reversed covalent bonds in the biomolecules of the sample, where such covalent bonds were previously formed by treatment with a fixation agent (e.g., paraformaldehyde or PFA).

Proteases are commonly used in processes for un-fixing fixed biological samples, usually in combination with un-fixing agents. The proteases break down proteins allowing un-fixing agents better access to the stabilizing crosslinks that are created by fixatives such as PFA. As noted above, however, the proteases needed to facilitate un-fixing of samples are often incompatible with the other enzymes used in partition-based assays, such as a nucleic acid processing enzyme (e.g., reverse transcriptase). The compositions, methods, and kits of the present disclosure that include a nano-partition capable of separating a first and second enzyme contained within the same partition can facilitate the use of fixed biological samples in partition-based assays.

Accordingly, in at least one embodiment the present disclosure provides a composition comprising a partition containing a fixed biological sample, an un-fixing agent, a first enzyme, wherein the first enzyme is a protease, a second enzyme, and a nano-partition, wherein the nano-partition separates the second enzyme from the biological sample and the first enzyme, thereby preventing the first and second enzymes from interacting. In at least one embodiment, the first enzyme is a protease and the second enzyme is an enzyme that is incompatible with a protease, e.g., that is inactivated rapidly by exposure to the protease. In at least one embodiment, the nano-partition encapsulates the second enzyme. In at least one embodiment, the nano-partition comprises pores having an average diameter of less than about 5 nm, less than about 4 nm, less than about 3.5 nm, less than about 3 nm, or less than about 2 nm. In at least one embodiment, the nano-partition comprises pores having an average diameter of between about 0.1 nm and about 10 nm, about 0.1 nm and about 5 nm, about 0.1 nm and about 3.5 nm, about 0.1 nm and about 2.5 nm, about 0.1 nm and about 2 nm, about 0.5 nm and about 10 nm, about 1 nm and about 8 nm, about 1.5 nm and about 6 nm, about 2 nm and about 5 nm, or about 2.3 nm and about 4 nm. In at least one embodiment, the nano-partition comprises pores that allow the diffusion of nucleic acids, such as mRNA molecules. In at least one embodiment, the nano-partition is also degradable by a stimulus, such as a stimulus selected from heat, UV light, and a chemical reagent.

Suitable proteases for use in the compositions and methods of the present disclosure include serine proteases (E.C. 3.4.21), such as the chymotrypsin-like, trypsin-like, thrombin-like, elastase-like, and subtilisin-like proteases. A wide range of different serine proteases are well-characterized and commercially available. Serine proteases useful in the compositions and methods of the present disclosure include, but are not limited to: alcalase, alkaline proteinase, ArcticZymes Proteinase (commercially available from: ArcticZymes Technologies ASA, Tromsø, Norway), bacillopeptidase A, bacillopeptidase B, bioprase, colistinase, esperase, genenase, kazusase, maxatase, pepsin, Serratia peptidase, proteinase K, protease S, savinase, subtilisin A, subtilisin B, subtilisin BL, subtilisin E, subtilisin J, subtilisin S, subtilisin S41, thermoase, and trypsin. It is further contemplated that mixtures these proteases may be used. Accordingly, in at least one embodiment, the compositions and methods of the present disclosure comprise a protease selected from alcalase, alkaline proteinase, ArcticZymes Proteinase, bacillopeptidase A, bacillopeptidase B, bioprase, colistinase, esperase, genenase, kazusase, maxatase, Serratia peptidase, proteinase K, protease S, savinase, subtilisin A, subtilisin B, subtilisin BL, subtilisin E, subtilisin J, subtilisin S, subtilisin S41, thermoase, trypsin, and a combination thereof. In at least one embodiment, the protease is selected from Subtilisin A, Proteinase K, ArcticZymes Proteinase, and a combination thereof.

In at least one embodiment, the present disclosure also provides a method for preparing such a composition comprising: generating a partition (e.g., a discrete droplet) encapsulating a fixed biological sample, an un-fixing agent, a first enzyme, wherein the first enzyme is a protease, a second enzyme, and a nano-partition, wherein the nano-partition separates the second enzyme from the biological sample and the first enzyme. In at least one embodiment, the nano-partition encapsulates the second enzyme. In at least one embodiment, the nano-partition comprises pores having an average diameter of less than about 5 nm, less than about 4 nm, less than about 3.5 nm, less than about 3 nm, or less than about 2 nm. In at least one embodiment, the nano-partition comprises pores having an average diameter of between about 0.1 nm and about 10 nm, about 0.1 nm and about 5 nm, about 0.1 nm and about 3.5 nm, about 0.1 nm and about 2.5 nm, about 0.1 nm and about 2 nm, about 0.5 nm and about 10 nm, about 1 nm and about 8 nm, about 1.5 nm and about 6 nm, about 2 nm and about 5 nm, or about 2.3 nm and about 4 nm. In at least one embodiment, the nano-partition comprises pores of a size that allow the diffusion of nucleic acids, such as mRNA molecules. In at least one embodiment, the nano-partition is also degradable by a stimulus, such as a stimulus selected from heat, UV light, and a chemical reagent.

In another embodiment, the present disclosure also provides partition-based assay methods using a nano-partition to separate two incompatible enzymes. “Incompatible enzymes” as used herein refers to at least two enzymes, having different catalytic activities, that when present in the same solution deleteriously effect the function of at least one of the enzymes. That is, the presence of at least one of the enzymes degrades, reduces, reverses, and/or inhibits the activity of the other enzyme, and/or degrades a substrate or product of a reaction catalyzed by the other enzyme. Proteases represent the best known class of enzymes that can exhibit incompatibility with other enzymes due to their proteolytic activity rapidly degrading the polypeptide structure and thus, function, of other enzymes in the same solution. For example, proteases and nucleic acid processing enzymes (e.g., polymerase, a cas enzyme, a restriction enzyme, a USER enzyme, a transposase, a ligase, a DNase, a reverse transcriptase (RT) enzymes) are incompatible enzymes, and when combined in a solution, the protease rapidly degrades their nucleic acid processing activity. Similarly, RNAse enzymes and ribozymes are incompatible because the RNAse activity degrades the structure of the RNA molecule that is essential for its catalytic function. Incompatible enzymes also can include any combination enzymes that have reverse or opposing catalytic functions, such as glycosidases and glycosyl transferases. The glycosyl transferase activity creates bonds to sugars, whereas the glycosidase cleaves such bonds, thereby reducing the apparent activity of the glycosyl transferase. Other examples of incompatible enzymes will be recognized by those of skill in the art, and the present disclosure contemplates that the nano-partition compositions and methods of can be used with any such enzymes based on its ability to separate one enzyme in a solution from another.

In at least one embodiment, the assay method comprises at least the steps of (a) generating a partition containing a biological sample, a first enzyme, a second enzyme, assay reagents, and a nano-partition, wherein the nano-partition separates the second enzyme from the biological sample and the first enzyme, thereby preventing the first and second enzymes from interacting; and (b) detecting analytes from the reaction of the biological sample, the assay reagents and the second enzyme type. In one embodiment, the biological sample undergoes a reaction with the first enzyme prior to step (b), and the products of that reaction are capable of diffusing through the porous material of the nano-partition and interacting with the second enzyme, which interaction typically results in analytes that are detected (e.g., cDNA molecules produced by reverse transcription of mRNA). As with the other methods utilizing nano-partitions described herein, in some embodiments, the nano-partition fully encapsulates the second enzyme. In some embodiments of the assay method, the nano-partition comprises pores having an average diameter of between about 0.5 nm and about 10 nm, and/or the nano-partition pores allow the diffusion of nucleic acids, such as mRNA molecules. In at least one embodiment, the nano-partition comprises pores having an average diameter of less than about 5 nm, less than about 4 nm, less than about 3.5 nm, less than about 3 nm, or less than about 2 nm. In at least one embodiment, the nano-partition pores have an average diameter of between about 0.1 nm and about 10 nm, about 0.1 nm and about 5 nm, about 0.1 nm and about 3.5 nm, about 0.1 nm and about 2.5 nm, about 0.1 nm and about 2 nm, about 0.5 nm and about 10 nm, about 1 nm and about 8 nm, about 1.5 nm and about 6 nm, about 2 nm and about 5 nm, or about 2.3 nm and about 4 nm.

It is also contemplated that the assay methods can be carried out using a nano-partition that is degradable by a stimulus, such as a stimulus selected from heat, UV light, and a chemical reagent. Accordingly, in at least one embodiment of the assay methods a degradable nano-partition is used and the method comprises (a) generating a partition containing a fixed biological sample, an un-fixing agent, assay reagents, a first enzyme, wherein the first enzyme is a protease, a second enzyme, and a degradable nano-partition, wherein the degradable nano-partition separates the second enzyme from the biological sample and the first enzyme; (b) providing a stimulus (e.g., heat, UV light, chemical) that degrades the nano-partition; and (c) detecting analytes from the reaction of the assay reagents and the un-fixed biological sample. In at least one embodiment, the first enzyme is a protease and the second enzyme is an enzyme that is incompatible with a protease, e.g., that is inactivated rapidly by exposure to the protease. Accordingly, it is contemplated that the first enzyme is inactivated by some means prior to, or simultaneously with application of the stimulus that degrade the nano-partition containing the second enzyme. In at least one embodiment, the stimulus applied is heat, and the heat acts to both inactivate the first enzyme, and to degrade the nano-partition.

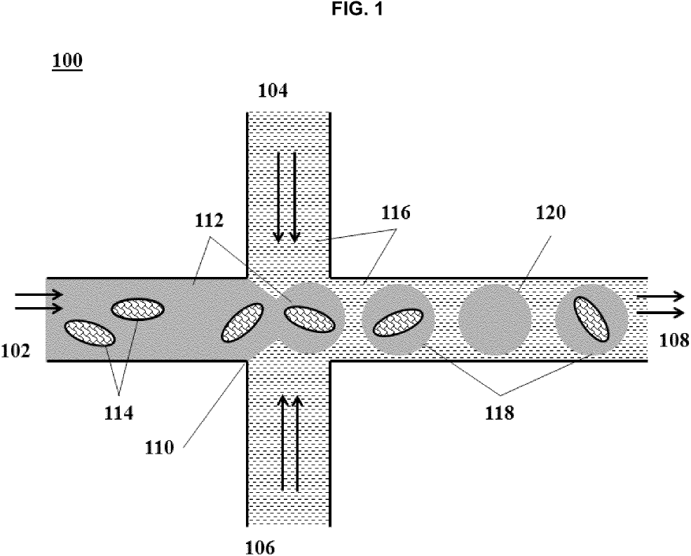

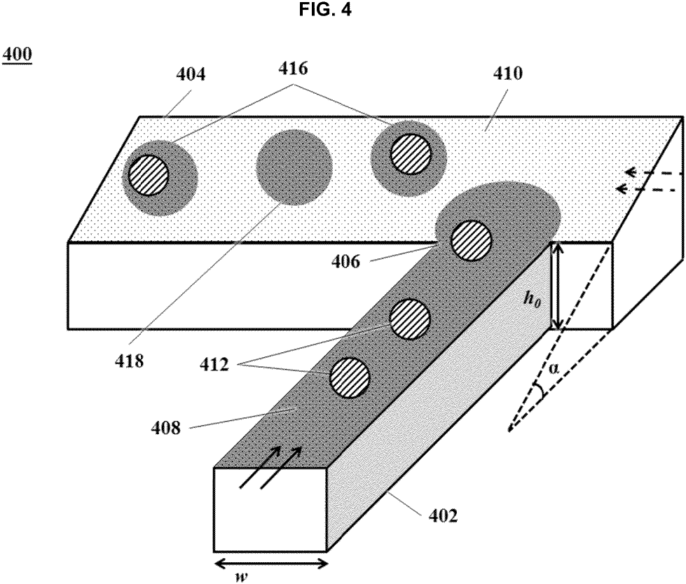

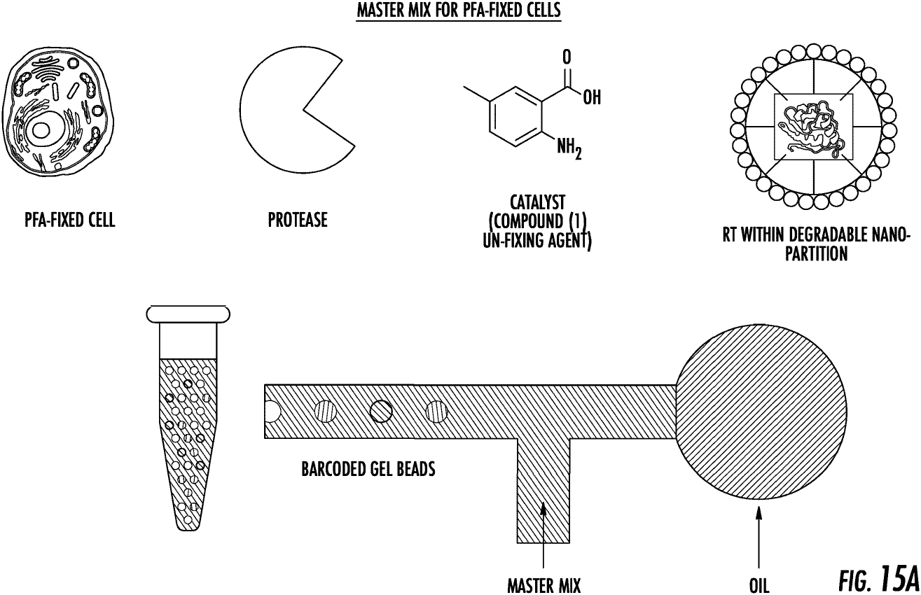

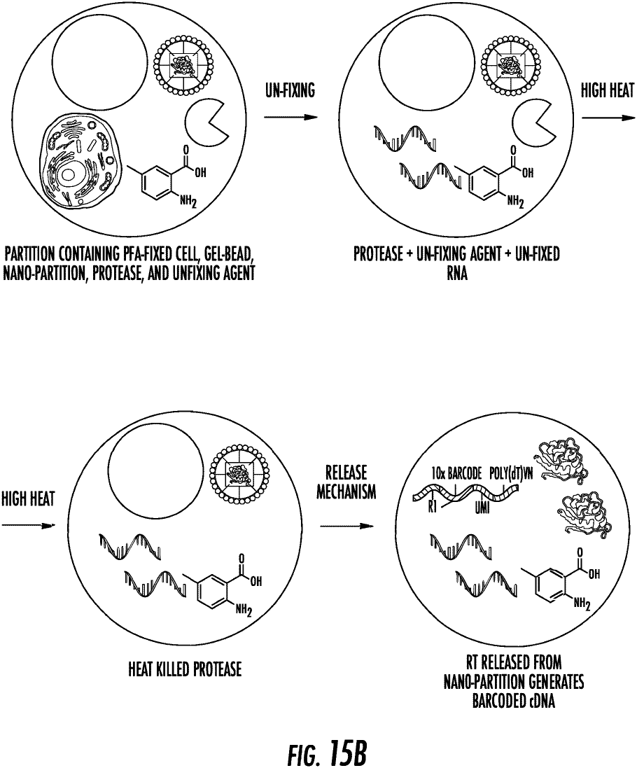

FIG. 15A and FIG. 15B provide a schematic depiction of an exemplary partition-based assay method using a degradable nano-partition. In the assay method depicted in FIG. 15A, a partition is generated containing a fixed biological particle, a protease, a catalytic un-fixing agent compound, a degradable nano-partition containing an RT enzyme, a barcoded gel bead (GEM), and optionally, other assay reagents. The partition may be generated according to a controlled partitioning method disclosed herein (e.g., as in FIG. 4). For example, the partition may be generated by: combining barcoded gel beads with a master mix comprising a fixed biological sample, the protease, the catalytic un-fixing agent, and the degradable nano-partition; and forming droplets at a junction with an oil reservoir (e.g., as depicted in FIG. 4). While FIG. 15A depicts droplet formation according to a controlled partitioning method, other methods and systems for droplet generation (e.g., as depicted in FIGS. 1-6) may be utilized. As depicted in FIG. 15B, upon formation of the droplet, the protease and un-fixing begin acting on the fixed biological sample to lyse the cell(s), release the cellular analytes of interest (e.g., mRNA) and reverse the fixative induced crosslinks between the analyte biomolecules that stabilize them but prevent the assay process. After the initial un-fixing step, heat is applied to the droplet that inactivates the protease while simultaneously degrading the nano-partition to release the RT enzyme. The RT enzyme (and other assay reagents such as primers, switch oligos, and NTPs) are then able to carry out the reverse transcription that generates cDNA molecules. Additionally, the RT can incorporate the oligonucleotide barcode sequences into the cDNA allowing for the ability to later trace the specific cellular analytes from this specific droplet.

The compositions and methods of the present disclosure allow for the use of fixed biological samples derived from a tissue sample, a biopsy sample, or a blood sample, that have been fixed with paraformaldehyde, and can comprise a fixed biological sample of a single cell, an organelle of a single cell, and/or a nucleus of a single cell. The stabilizing effect of the fixatives together with the efficient use of proteases and un-fixing agents disclosed herein allow for the amount of time of sample fixation prior to generating the discrete droplet to be at least 1 hour, at least 2 hours, at least 6 hours, at least 12 hours, at least 24 hours, at least 1 week, at least 1 month, at least 6 months, or longer.

The amount of time a biological sample is contacted with a fixative to provide a fixed biological sample depend on the temperature, the nature of the sample, and the fixative used. For example, a biological sample can be contacted by a fixation reagent for 72 or less hours (e.g., 48 or less hours, 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes).

Generally, contact of biological sample (e.g., a cell) with a fixation reagent (e.g., paraformaldehyde or PFA) results the formation of intra- and inter-molecular covalent crosslinks between biomolecules in the biological sample. In some cases, the fixation reagent, formaldehyde, is known to result in covalent aminal crosslinks within RNA, DNA, and/or protein molecules. Examples of fixation reagents include but are not limited to aldehyde fixatives (e.g., formaldehyde, also commonly referred to as “paraformaldehyde,” “PFA,” and “formalin”; glutaraldehyde; etc.), imidoesters, NHS (N-Hydroxysuccinimide) esters, and the like.

The formation of crosslinks in biomolecules (e.g., proteins, RNA, DNA) due to fixation greatly reduces the ability to detect (e.g., bind to, amplify, sequence, hybridize to) the biomolecules in standard assay methods. Common techniques to remove the crosslinks induced by fixative reagents (e.g., heat, acid) can cause further damage to the biomolecules (e.g., loss of bases, chain hydrolysis, cleavage, denaturation, etc.). Further description of the consequences of fixation of tissue samples and the benefits of removing adducts and/or crosslinks are described in US Pat. No. 8,288,122, which is hereby incorporated by reference in its entirety. For example, the widely used fixative reagent, paraformaldehyde or PFA, fixes tissue samples by catalyzing crosslink formation between basic amino acids in proteins, such as lysine and glutamine. Both intra-molecular and inter-molecular crosslinks can form in the protein. These crosslinks can preserve protein secondary structure and also eliminate enzymatic activity in the preserved tissue sample.

In some embodiments, the fixative or fixation reagent useful in the methods of the present disclosure is formaldehyde. The term “formaldehyde” when used in the context of a fixative also refers “paraformaldehyde” (or “PFA”) and “formalin”, both of which are terms with specific meanings related to the formaldehyde composition (e.g., formalin is a mixture of formaldehyde and methanol). Thus, a formaldehyde-fixed biological sample may also be referred to as formalin-fixed or PFA-fixed. Protocols and methods for the use of formaldehyde as a fixation reagent to prepare fixed biological samples are well known in the art, and can be used in the methods and compositions of the present disclosure. For example, suitable ranges of formaldehyde concentrations for use in preparing a fixed biological sample is 0.1% to 10%, 1% to 8%, 1% to 4%, 1% to 2%, 3% to 5%, or 3.5% to 4.5%. In some embodiments of the present disclosure the biological sample is fixed using a final concentration of 1 % formaldehyde, 4% formaldehyde, or 10% formaldehyde. Typically, the formaldehyde is diluted from a more concentrated stock solution - e.g., a 35%, 25%, 15%, 10%, 5% PFA stock solution.

It is contemplated that more than one fixation reagent can be used in combination in preparing a fixed biological sample. For example, in some cases biomolecules (e.g., biological samples such as tissue specimens) are contacted with a fixation reagent containing both formaldehyde and glutaraldehyde, and thus the contacted biomolecules can include fixation crosslinks resulting both from formaldehyde induced fixation and glutaraldehyde induced fixation. Typically, a suitable concentration of glutaraldehyde for use as a fixation reagent is 0.1 to 1%.

Conditions for reversing the effects of fixing a biological sample are known in the art, however, these conditions tend to be harsh. See e.g., PCT Publ. No. WO2001/46402; U.S. Pat. Publ. Nos. 2005/0014203A1, and 2009/0202998A1, each of which is hereby incorporated by reference herein. For example, treatment of PFA-treated tissue samples includes heating to 60-70C in Tris buffer for several hours, and yet typically results in removal of only a fraction of the fixative-induced crosslinks. Furthermore, the harsh un-fixing treatment conditions can result in permanent damage to biomolecules, particularly nucleic acids, in the sample. Recently, less harsh un-fixing techniques and conditions have been proposed that utilize compounds capable of chemically reversing the crosslinks resulting from fixation. See e.g., Karmakar et al., “Organocatalytic removal of formaldehyde adducts from RNA and DNA bases,” Nature Chemistry, 7: 752-758 (2015); U.S. Pat. Publ. Nos. 2017/0283860A1 and 2019/0135774A1, each of which is hereby incorporated by reference herein.

The term “un-fixing agent” (or “de-crosslinking agent”) as used herein refers to a compound or composition that reverses fixation and/or removes the crosslinks within or between biomolecules in a sample caused by previous use of a fixation reagent. In some embodiments, un-fixing agents are compounds that act catalytically in removing crosslinks in a fixed sample. Exemplary compounds useful as un-fixing agents in the methods and compositions of the present disclosure include the compounds of Table 1 below.

TABLE 1

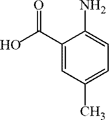

(1) 2-amino-5-methylbenzoic acid

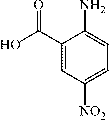 [SMILES: Nc1ccc([N+](=O)[O-])cc1C(=O)O]

(2) 2-amino-5-nitrobenzoic acid

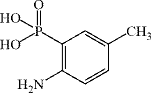

(3) (2-amino-5-methylphenyl)phosphonic acid

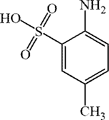

(4) 2-amino-5-methylbenzenesulfonic acid

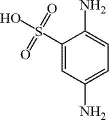 [SMILES: Nc1ccc(N)c(S(=O)(=O)O)c1]

(5) 2,5-diaminobenzenesulfonic acid

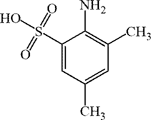

(6) 2-amino-3,5-dimethylbenzenesulfonic acid

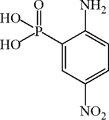 [SMILES: Nc1ccc([N+](=O)[O-])cc1P(=O)(O)O]

(7) (2-amino-5-nitrophenyl)phosphonic acid

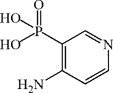 [SMILES: Nc1ccncc1P(=O)(O)O]

(8) (4-aminopyridin-3-yl)phosphonic acid

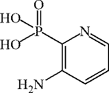 [SMILES: Nc1cccnc1P(=O)(O)O]

(9)

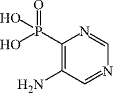 [SMILES: Nc1cncnc1P(=O)(O)O]

(10)

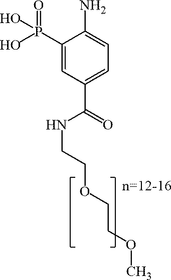

(11) (2-amino-5-{[2-(2-poly-ethoxy)ethyl]carbamoyl}phenyl)phosphonic acid

Accordingly, in some embodiments of the compositions and methods of the present disclosure, the un-fixing agent used in the composition or method can comprise a compound selected from Table 1. For example, the un-fixing agent can comprise a compound of any of compound (1), compound (2), compound (3), compound (4), compound (5), compound (6), compound (7), compound (8), compound (9), compound (10), compound (11), or a combination of one or more the compounds of Table 1. Without intending to be limited by any particular chemical mechanism, it is believed that the compounds of Table 1 catalytically break down the aminal and hemi-aminal adducts that form in RNA treated with formaldehyde, and are compatible with many RNA extraction and detection conditions. This type of mechanism has been described previously for Compound (3) (Karmakar et al., “Organocatalytic removal of formaldehyde adducts from RNA and DNA bases,” Nature Chemistry, 7: 752-758 (2015); and U.S. Pat. Publ. No. 2017/0283860A1, which is hereby incorporated by reference herein).

### C. Generating Partitions Containing Biological Samples, Enzymes, and Nano-Partitions

The compositions and methods of the present disclosure comprising nano-partitions containing an enzyme are useful to prepare biological samples partitioned in discrete droplets along with a biological sample, other enzymes, and reagents and components useful in partition-based methods and assays. Accordingly, in at least one embodiment, the present disclosure provides a method for preparing a biological sample wherein the method comprises generating a partition containing a biological sample, a first enzyme, a second enzyme, and a nano-partition, wherein the nano-partition separates the second enzyme from the biological sample and the first enzyme, thereby preventing the first and second enzymes from interacting. In at least one embodiment, the partition is a discrete droplet.

Methods, techniques, and protocols useful for generating biological samples (e.g., individual cells, biomolecular contents of cells, etc.) in partitions, such as discrete droplets, are described in the art. The discrete droplet partitions generated act a nanoliter-scale container that can maintain separation of the droplet contents from the contents of other droplets in an immiscible emulsion. Materials, methods and systems for creating stable discrete droplets encapsulating a biological sample in non-aqueous or oil emulsions are described in, e.g., U.S. Pat. Publ. Nos. 2010/0105112A1 and 2019/0100632A1, each of which is entirely incorporated herein by reference for all purposes.

Briefly, discrete droplets in an emulsion encapsulating a biological sample is accomplished by introducing a flowing stream of an aqueous fluid containing the biological sample into a flowing stream of a non-aqueous fluid with which it is immiscible, such that droplets are generated at the junction of the two streams (see e.g., FIGS. 1-3). By providing the aqueous stream at a certain concentration and/or flow rate of the biological sample, the occupancy of the resulting droplets can be controlled. For example, the relative flow rates of the immiscible fluids can be selected such that, on average, the discrete droplet each contains less than one cell or particle of a biological sample. Such a flow rate ensures that the droplets that are occupied are primarily occupied by a single sample (e.g., a single cell, an organelle of a single cell and/or a nuclei of a single cell). Discrete droplets in an emulsion encapsulating a biological sample is also accomplished using a microfluidic architecture comprising a channel segment having a channel junction with a reservoir (see FIGS. 4-6). In some cases, the droplets among a plurality of discrete droplets formed in the manner contain at most one biological sample (e.g., one cell). The flows and microfluidic channel architectures also can be controlled to ensure a given number of singly occupied droplets, less than a certain level of unoccupied droplets, and/or less than a certain level of multiply occupied droplets.

FIG. 1 shows an exemplary microfluidic channel structure 100 useful for generating discrete droplets encapsulating a cell or particle of a biological sample. The channel structure 100 can include channel segments 102, 104, 106 and 108 communicating at a channel junction 110. In operation, a first aqueous fluid 112 that that includes suspended cell or particle of a biological sample 114 are transported along channel segment 102 into junction 110, while a second fluid 116 (or “partitioning fluid”) that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 may be fluidically coupled to an outlet reservoir where the discrete droplets can be stored and/or harvested. A discrete droplet generated may include an individual cell or particle from a biological sample 114 (such as droplet 118), or discrete droplet can be generated that includes more than one cell or particle 114 (not shown in FIG. 1). A discrete droplet may contain no biological sample 114 (such as droplet 120). Each discrete droplet is capable of maintaining separation of its own contents (e.g., individual biological sample 114) from the contents of other droplets.

Typically, the second fluid 116 comprises an oil, such as a fluorinated oil, that includes a fluoro-surfactant that helps to stabilize the resulting droplets. Examples of useful partitioning fluids and fluoro-surfactants are described in e.g., U.S. Pat. Publ. No. 2010/0105112A1, which is entirely incorporated herein by reference for all purposes.

The microfluidic channels for generating discrete droplets as exemplified in FIG. 1 may be coupled to any of a variety of different fluid sources or receiving components, including reservoirs, tubing, manifolds, or fluidic components of other systems. Additionally, the microfluidic channel structure 100 may have other geometries, including geometries having more than one channel junction. For example, the microfluidic channel structure can have 2, 3, 4, or 5 channel segments each carrying a biological sample, assay reagents, and/or beads that meet at a channel junction.

Generally, the fluids used in generating the discrete droplets are directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electro-kinetic pumping, vacuum, capillary or gravity flow, or the like.

One of ordinary skill will recognize that numerous different microfluidic channel designs and methods known in the art for generating partitions (e.g., a discrete droplet) containing a biological sample, a bead, and/or assay reagents, can also be used with the methods and compositions of the present disclosure to generate partitions that further contain a nano-partition. Thus, a composition of the present disclosure comprising a partition containing a biological sample, a first enzyme, a second enzyme, and a nano-partition, wherein the nano-partition separates the second enzyme from the biological sample and the first enzyme, can be generated with using the microfluidic channel designs and systems described herein, and elsewhere in the art.

In at least one embodiment, the biological sample may be partitioned with a support (e.g., a bead) comprising nucleic acid molecules suitable for barcoding of the one or more analytes. In another embodiment, the nucleic acid molecules may include nucleic acid sequences that provide identifying information, e.g., barcode sequence(s). The inclusion of a barcode in a partition along with the biological sample provides a unique identifier that allows data from the biological sample to be distinguished and individually analyzed. The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

As used herein, the term “barcoded nucleic acid molecule” generally refers to a nucleic acid molecule that results from, for example, the processing of a nucleic acid barcode molecule with a nucleic acid sequence (e.g., nucleic acid sequence complementary to a nucleic acid primer sequence encompassed by the nucleic acid barcode molecule). The nucleic acid sequence may be a targeted sequence (e.g., targeted by a primer sequence) or a non-targeted sequence. For example, in the methods, compositions, kits, and systems described herein, hybridization and reverse transcription of the nucleic acid molecule (e.g., an mRNA molecule) of a cell with a nucleic acid barcode molecule (e.g., a nucleic acid barcode molecule containing a barcode sequence and a nucleic acid primer sequence complementary to a nucleic acid sequence of the mRNA molecule) results in a barcoded nucleic acid molecule that has a sequence corresponding to the nucleic acid sequence of the mRNA and the barcode sequence (or a reverse complement thereof). A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the mRNA.

It is contemplated that in the methods of the present disclosure that barcodes can be delivered into the partition previous to, subsequent to, or concurrent with the biological sample, the nano-partition, the first enzyme, and/or other assay reagents. For example, barcodes may be injected into an aqueous mixture used to form a discrete droplet previous to droplet formation in an immiscible oil phase. Barcodes useful in the methods and compositions of the present disclosure typically comprise a nucleic acid molecule (e.g., an oligonucleotide). The nucleic acid molecules typically are delivered to a partition via a solid or semi-solid phase, such as a bead, to which the barcode molecules are linked. In some cases, barcode nucleic acid molecules are initially associated with a bead upon generation of the discrete droplet, and then released from the bead upon application of a stimulus to droplet. Barcode carrying beads useful in the methods and compositions of the present disclosure are described in further detail elsewhere herein.

Methods and systems for partitioning barcode carrying beads into droplets are provided in U.S. Pat. Nos. 10480029, 10858702, and 10725027, U.S. Pat. Publication Nos. 2019/0367997 and 2019/0064173, and International Application Nos. PCT/US20/17785 and PCT/US20/020486, each of which is herein entirely incorporated by reference for all purposes.

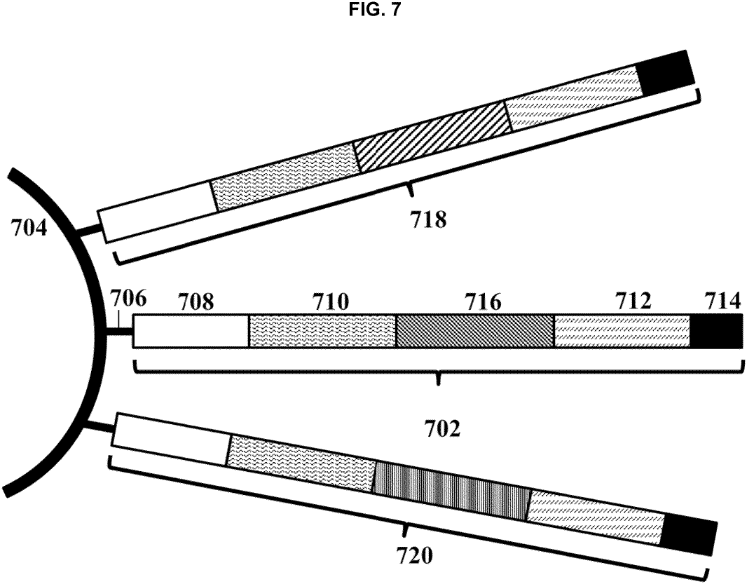

FIG. 7 illustrates an example of a barcode carrying bead. A nucleic acid molecule 702, such as an oligonucleotide, can be coupled to a bead 704 by a releasable linkage 706, such as, for example, a disulfide linker. The same bead 704 may be coupled (e.g., via releasable linkage) to one or more other nucleic acid molecules 718, 720. The nucleic acid molecule 702 may be or comprise a barcode. As noted elsewhere herein, the structure of the barcode may comprise a number of sequence elements. The nucleic acid molecule 702 may comprise a functional sequence 708 that may be used in subsequent processing. For example, the functional sequence 708 may include one or more of a sequencer specific flow cell attachment sequence (e.g., a P5 sequence for Illumina® sequencing systems) and a sequencing primer sequence (e.g., a R1 primer for Illumina® sequencing systems). The nucleic acid molecule 702 may comprise a barcode sequence 710 for use in barcoding the sample (e.g., DNA, RNA, protein, antibody, etc.). In some cases, the barcode sequence 710 can be bead-specific such that the barcode sequence 710 is common to all nucleic acid molecules (e.g., including nucleic acid molecule 702) coupled to the same bead 704. Alternatively or in addition, the barcode sequence 710 can be partition-specific such that the barcode sequence 710 is common to all nucleic acid molecules coupled to one or more beads that are partitioned into the same partition. The nucleic acid molecule 702 may comprise a specific priming sequence 712, such as an mRNA specific priming sequence (e.g., poly-T sequence), a targeted priming sequence, and/or a random priming sequence. The nucleic acid molecule 702 may comprise an anchoring sequence 714 to ensure that the specific priming sequence 712 hybridizes at the sequence end (e.g., of the mRNA). For example, the anchoring sequence 714 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid molecule 702 may comprise a unique molecular identifying sequence 716 (e.g., unique molecular identifier (UMI)). In some cases, the unique molecular identifying sequence 716 may comprise from about 5 to about 8 nucleotides. Alternatively, the unique molecular identifying sequence 716 may compress less than about 5 or more than about 8 nucleotides. The unique molecular identifying sequence 716 may be a unique sequence that varies across individual nucleic acid molecules (e.g., 702, 718, 720, etc.) coupled to a single bead (e.g., bead 704). In some cases, the unique molecular identifying sequence 716 may be a random sequence (e.g., such as a random N-mer sequence). For example, the UMI may provide a unique identifier of the starting mRNA molecule that was captured, in order to allow quantitation of the number of original expressed RNA. As will be appreciated, although FIG. 7 shows three nucleic acid molecules 702, 718, 720 coupled to the surface of the bead 704, an individual bead may be coupled to any number of individual nucleic acid molecules, for example, from one to tens to hundreds of thousands or even millions of individual nucleic acid molecules. The respective barcodes for the individual nucleic acid molecules can comprise both common sequence segments or relatively common sequence segments (e.g., 708, 710, 712, etc.) and variable or unique sequence segments (e.g., 716) between different individual nucleic acid molecules coupled to the same bead.

A biological particle (e.g., cell, fixed cell, un-fixed cell, organelle, fixed organelle, unfixed organelle, nucleus of a cell, fixed nucleus of a cell, unfixed nucleus of a cell, DNA, RNA, etc.) can be co-partitioned along with a barcode bearing bead 704. The barcoded nucleic acid molecules 702, 718, 720 can be released from the bead 704 in the partition. By way of example, in the context of analyzing sample RNA, the poly-T segment (e.g., 712) of one of the released nucleic acid molecules (e.g., 702) can hybridize to the poly-A tail of a mRNA molecule. Reverse transcription may result in a cDNA transcript of the mRNA, but which transcript includes each of the sequence segments 708, 710, 716 of the nucleic acid molecule 702. Because the nucleic acid molecule 702 comprises an anchoring sequence 714, it will more likely hybridize to and prime reverse transcription at the sequence end of the poly-A tail of the mRNA. Within any given partition, all of the cDNA transcripts of the individual mRNA molecules may include a common barcode sequence segment 710.

However, the transcripts made from the different mRNA molecules within a given partition may vary at the unique molecular identifying sequence 712 segment (e.g., UMI segment). Beneficially, even following any subsequent amplification of the contents of a given partition, the number of different UMIs can be indicative of the quantity of mRNA originating from a given partition, and thus from the biological particle (e.g., cell, fixed cell, un-fixed cell, organelle, fixed organelle, unfixed organelle, nucleus of a cell, fixed nucleus of a cell, unfixed nucleus of a cell, etc.). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid molecules bound to the bead (e.g., gel bead) may be used to hybridize and capture the mRNA on the solid phase of the bead, for example, in order to facilitate the separation of the RNA from other cell contents. In such cases, further processing may be performed, in the partitions or outside the partitions (e.g., in bulk). For instance, the RNA molecules on the beads may be subjected to reverse transcription or other nucleic acid processing, additional adapter sequences may be added to the barcoded nucleic acid molecules, or other nucleic acid reactions (e.g., amplification, nucleic acid extension) may be performed. The beads or products thereof (e.g., barcoded nucleic acid molecules) may be collected from the partitions, and/or pooled together and subsequently subjected to clean up and further characterization (e.g., sequencing). The operations described herein may be performed at any useful or convenient step. For instance, the beads comprising nucleic acid barcode molecules may be introduced into a partition (e.g., well or droplet) prior to, during, or following introduction of a sample into the partition. The nucleic acid molecules of a sample may be subjected to barcoding, which may occur on the bead (in cases where the nucleic acid molecules remain coupled to the bead) or following release of the nucleic acid barcode molecules into the partition. In cases where the nucleic acid molecules from the sample remain attached to the bead, the beads from various partitions may be collected, pooled, and subjected to further processing (e.g., reverse transcription, adapter attachment, amplification, clean up, sequencing). In other instances, the processing may occur in the partition. For example, conditions sufficient for barcoding, adapter attachment, reverse transcription, or other nucleic acid processing operations may be provided in the partition and performed prior to clean up and sequencing.

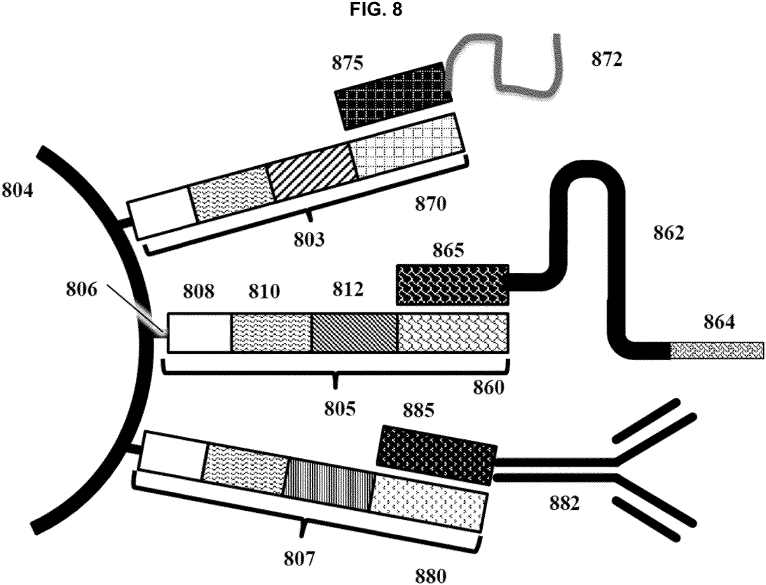

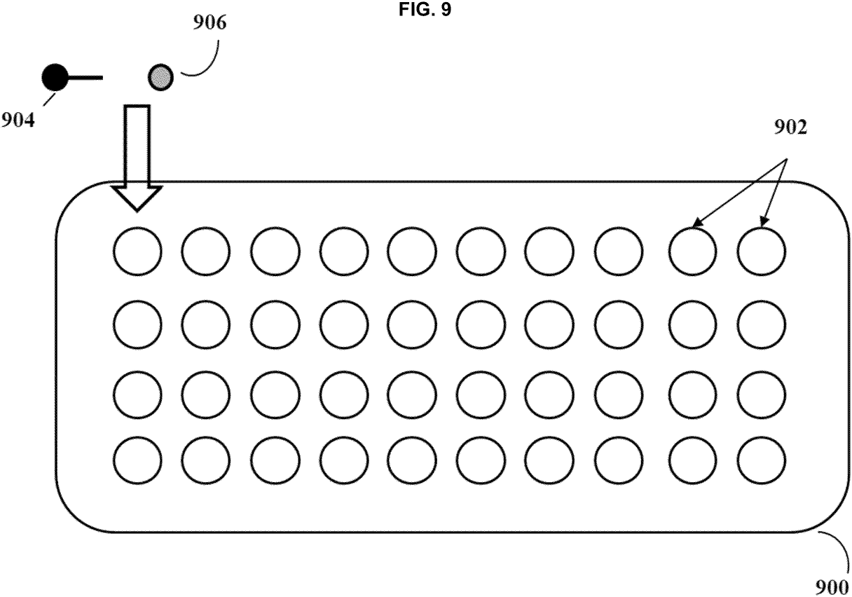

FIG. 8 illustrates another example of a barcode carrying bead. A nucleic acid molecule 805, such as an oligonucleotide, can be coupled to a bead 804 by a releasable linkage 806, such as, for example, a disulfide linker. The nucleic acid molecule 805 may comprise a first capture sequence 860. The same bead 804 may be coupled (e.g., via releasable linkage) to one or more other nucleic acid molecules 803, 807 comprising other capture sequences. The nucleic acid molecule 805 may be or comprise a barcode. As noted elsewhere herein, the structure of the barcode may comprise a number of sequence elements, such as a functional sequence 808 (e.g., flow cell attachment sequence, sequencing primer sequence, etc.), a barcode sequence 810 (e.g., bead-specific sequence common to bead, partition-specific sequence common to partition, etc.), and a unique molecular identifier 812 (e.g., unique sequence within different molecules attached to the bead), or partial sequences thereof. The capture sequence 860 may be configured to attach to a corresponding capture sequence 865. In some instances, the corresponding capture sequence 865 may be coupled to another molecule that may be an analyte or an intermediary carrier. For example, as illustrated in FIG. 8, the corresponding capture sequence 865 is coupled to a guide RNA molecule 862 comprising a target sequence 864, wherein the target sequence 864 is configured to attach to the analyte. Another oligonucleotide molecule 807 attached to the bead 804 comprises a second capture sequence 880 which is configured to attach to a second corresponding capture sequence 885. As illustrated in FIG. 8, the second corresponding capture sequence 885 is coupled to an antibody 882. In some cases, the antibody 882 may have binding specificity to an analyte (e.g., surface protein). Alternatively, the antibody 882 may not have binding specificity. Another oligonucleotide molecule 803 attached to the bead 804 comprises a third capture sequence 870 which is configured to attach to a second corresponding capture sequence 875. As illustrated in FIG. 8, the third corresponding capture sequence 875 is coupled to a molecule 872. The molecule 872 may or may not be configured to target an analyte. The other oligonucleotide molecules 803, 807 may comprise the other sequences (e.g., functional sequence, barcode sequence, UMI, etc.) described with respect to oligonucleotide molecule 805. While a single oligonucleotide molecule comprising each capture sequence is illustrated in FIG. 8, it will be appreciated that, for each capture sequence, the bead may comprise a set of one or more oligonucleotide molecules each comprising the capture sequence. For example, the bead may comprise any number of sets of one or more different capture sequences. Alternatively, or in addition, the bead 804 may comprise other capture sequences. Alternatively, or in addition, the bead 804 may comprise fewer types of capture sequences (e.g., two capture sequences). Alternatively or in addition, the bead 804 may comprise oligonucleotide molecule(s) comprising a priming sequence, such as a specific priming sequence such as an mRNA specific priming sequence (e.g., poly-T sequence), a targeted priming sequence, and/or a random priming sequence, for example, to facilitate an assay for gene expression.

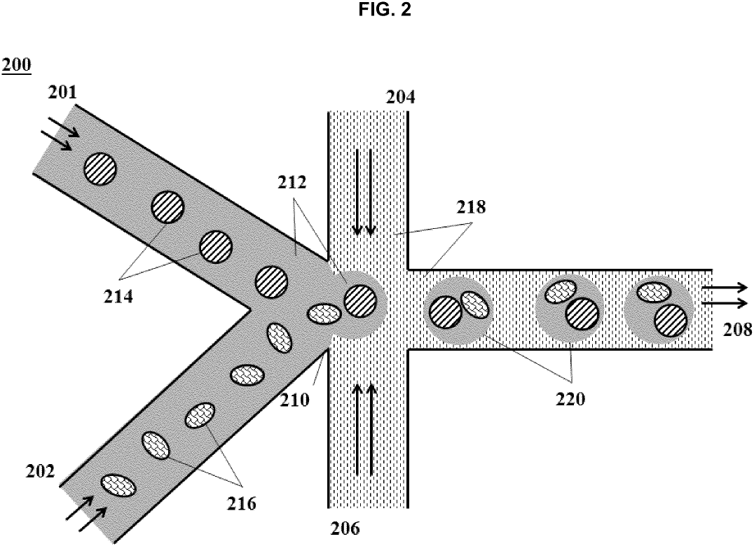

FIG. 2 shows an exemplary microfluidic channel structure 200 for generating discrete droplets encapsulating a barcode carrying bead 214 along with a biological sample 216. The channel structure 200 includes channel segments 201, 202, 204, 206 and 208 in fluid communication at a channel junction 210. In operation, the channel segment 201 transports an aqueous fluid 212 that can include a plurality of beads 214 (e.g., gel beads carrying barcode oligonucleotides) along the channel segment 201 into junction 210. The plurality of beads 214 may be sourced from a suspension of beads. For example, the channel segment 201 can be connected to a reservoir comprising an aqueous suspension of beads 214. The channel segment 202 transports the aqueous fluid 212 that includes a biological sample, such as a plurality of cells or particles 216 along the channel segment 202 into junction 210. The plurality of cells or particles 216 may be sourced from a suspension of the biological sample. For example, the channel segment 202 may be connected to a reservoir comprising an aqueous suspension of the biological sample 216. In some instances, the aqueous fluid 212 in either the first channel segment 201 or the second channel segment 202, or in both segments, can include one or more reagents, as further described elsewhere herein. For example, in some embodiments of the present disclosure, where the biological sample is a fixed biological sample, the aqueous fluid in the first and/or second channel segments that delivers the biological sample and beads, respectively, can include an un-fixing agent. The second fluid 218 that is immiscible with the aqueous fluid 212 is delivered to the junction 210 from each of channel segments 204 and 206. Upon meeting of the aqueous fluid 212 from each of channel segments 201 and 202 and the second fluid 218 (e.g., a fluorinated oil) from each of channel segments 204 and 206 at the channel junction 210, the aqueous fluid 212 is partitioned into discrete droplets 220 in the second fluid 218 and flow away from the junction 210 along channel segment 208. The channel segment 208 can then deliver the discrete droplets encapsulating the biological sample and a barcode bead to an outlet reservoir fluidly coupled to the channel segment 208, where they can be collected.

As an alternative, the channel segments 201 and 202 may meet at another junction upstream of the junction 210. At such junction, beads and biological particles may form a mixture that is directed along another channel to the junction 210 to yield droplets 220. The mixture may provide the beads and biological sample in an alternating fashion, such that, for example, a droplet comprises a single bead and a single biological sample cell or particle.

Using such a channel system as exemplified in FIG. 2, discrete droplets 220 can be generated that encapsulate an individual biological sample, and one bead, wherein the bead can carry a barcode and/or another reagent. It is also contemplated, that in some instances, a discrete droplet may be generated using the channel system of FIG. 2, wherein droplet includes more than one individual biological sample or includes no biological sample. Similarly, in some embodiments, the discrete droplet may include more than one bead or no bead. A discrete droplet also may be completely unoccupied (e.g., no bead or biological sample).

In some embodiments, it is desired that the beads, biological sample, and generated discrete droplets flow along channels at substantially regular flow rates that generate a discrete droplet containing a single bead and a single biological sample particle. Regular flow rates and devices that may be used to provide such regular flow rates are known in the art, and described in e.g., U.S. Pat. Publ. No. 2015/0292988A1, which is hereby incorporated by reference herein in its entirety. In some embodiments, the flow rates are set to provide discrete droplets containing a single bead and a biological sample with a yield rate of greater than 5%, 10%, 20%, 30%, 40%, 50%, 60%, 70%, 80%, 90%, or 95%.

### D. Supports

Supports, such as beads, that can carry barcodes and/or other reagents are useful with the methods of the present disclosure and can include, without limitation, supports that are porous, non-porous, solid, semi-solid, semi-fluidic, fluidic, and/or a combination thereof. In some embodiments, the support is a bead that is made of a material that is dissolvable, disruptable, and/or degradable, such as a gel bead comprising a hydrogel. Alternatively, in some embodiments, the support is not degradable.

In some embodiments of the present disclosure, the support is a bead that can be encapsulated in a discrete droplet with a biological sample. The term “bead,” as used herein, generally refers to a particle. Beads include solid or semi-solid particles, such as a gel bead. Typically, the bead useful in the embodiments disclosed herein comprise a hydrogel. Gel beads include a polymer matrix (e.g., matrix formed by polymerization or cross-linking). The polymer matrix may be made up of one or more different polymers (e.g., polymers having different functional groups or repeat units), and the polymers in the matrix may be randomly arranged, such as in random copolymers, and/or have ordered structures, such as in block copolymers. Cross-linking between polymers in the polymer matrix can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers, which may be natural or synthetic. Such polymers or monomers may be or include nucleic acid molecules (e.g., DNA or RNA). The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such a coating may be disruptable, degradable, or dissolvable. Beads useful with the compositions and methods can be of materials that are porous, non-porous, solid, semi-solid, semi-fluidic, fluidic, and/or a combination thereof. In some embodiments, the bead is a gel bead comprising a hydrogel matrix. Such gel beads can be formed from polymeric or monomeric precursor molecules that undergo a crosslinking reaction to form a hydrogel matrix. Another semi-solid bead useful in the present disclosure is a liposomal bead. In some embodiments, beads used can be solid beads that comprise a metal including iron oxide, gold, and silver. In some cases, the bead may be a silica bead. In some cases, the bead can be rigid. In other cases, the bead may be flexible and/or compressible. Generally, the beads can be of any suitable shape. Examples of bead shapes include, but are not limited to, spherical, non-spherical, oval, oblong, amorphous, circular, cylindrical, and variations thereof.

The beads useful in the methods and compositions of the present disclosure can comprise a range of natural and/or synthetic materials. For example, a bead can comprise a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads may also be formed from materials other than polymers, including lipids, micelles, ceramics, glass-ceramics, material composites, metals, other inorganic materials, and others.

The beads useful in the methods and compositions of the present disclosure can be of uniform size or they can comprise a collection of heterogeneous sizes. In some cases, the diameter of a bead is at least about 1 micron (µm), 5 µm, 10 µm, 20 µm, 30 µm, 40 µm, 50 µm, 60 µm, 70 µm, 80 µm, 90 µm, 100 µm, 250 µm, 500 µm, 1000 µm (1 mm), or greater. In some cases, a bead may have a diameter of less than about 1 µm, 5 µm, 10 µm, 20 µm, 30 µm, 40 µm, 50 µm, 60 µm, 70 µm, 80 µm, 90 µm, 100 µm, 250 µm, 500 µm, 1 mm, or less. In some cases, a bead may have a diameter in the range of about 40-75 µm, 30-75 µm, 20-75 µm, 40-85 µm, 40-95 µm, 20-100 µm, 10-100 µm, 1-100 µm, 20-250 µm, or 20-500 µm.

The beads used can comprise a population or plurality of individual beads having an overall relatively monodisperse size distribution. Typically, where it is desirable to provide a consistent amount of a bead-associated reagent within a partition, the use of relatively consistent bead characteristics, such as size, provides overall consistency in the content of each droplet. For example, the beads useful in the embodiments of the present disclosure can have size distributions that have a coefficient of variation in their cross-sectional dimensions of less than 50%, less than 40%, less than 30%, less than 20%, and in some cases less than 15%, less than 10%, less than 5%, or less.

Although the exemplary embodiments of FIG. 1 and FIG. 2 have been described in terms of providing discrete droplets that are predominantly singly occupied, it is also contemplated in certain embodiments that it is desirable to provide multiply occupied discrete droplets, e.g., a single droplet that contains two, three, four or more cells from a biological sample, and/or multiple different nano-partitions, or multiple different beads. For example, a partition containing two beads, one carrying a barcode nucleic acid molecule and one carrying a reagent such as an un-fixing agent or assay reagent. Accordingly, as noted elsewhere herein, the flow characteristics of the biological particle and/or the beads can be controlled to provide for such multiply occupied droplets. In particular, the flow parameters of the liquids used in the channel structures may be controlled to provide a given droplet occupancy rate greater than about 50%, greater than about 75%, and in some cases greater than about 80%, 90%, 95%, or higher.

In some embodiments, different beads (e.g., containing different reagents) can be introduced from different sources into different inlets leading to a common droplet generation junction (e.g., junction 210). In such cases, the flow and frequency of the different beads into the channel or junction may be controlled to provide for a certain ratio of beads from each source, while ensuring a given pairing or combination of such beads into a partition with a given number of biological particles (e.g., one cell and one bead per partition).

The discrete droplets described herein generally comprise small volumes, for example, less than about 10 microliters (µL), 5 µL, 1 µL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less. In some embodiments, the discrete droplets generated that encapsulate a biological sample particle have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. It will be appreciated that the sample fluid volume, e.g., including co-partitioned biological samples, nano-partitions, and/or beads, within the droplets may be less than about 90% of the above described volumes, less than about 80%, less than about 70%, less than about 60%, less than about 50%, less than about 40%, less than about 30%, less than about 20%, or less than about 10% of the above described volumes.

The methods of generating discrete droplets useful with the compositions and methods of the present disclosure, result in the generation of a population or plurality of discrete droplets containing a biological sample (e.g., a fixed biological sample), other reagents (e.g., a protease and an un-fixing agent), and a nano-partition carrying another enzyme (e.g., a reverse transcriptase). Generally, the methods are easily controlled to provide for any suitable number of droplets. For example, at least about 1,000 discrete droplets, at least about 5,000 discrete droplets, at least about 10,000 discrete droplets, at least about 50,000 discrete droplets, at least about 100,000 discrete droplets, at least about 500,000 discrete droplets, at least about 1,000,000 discrete droplets, at least about 5,000,000 discrete droplets, at least about 10,000,000 discrete droplets, or more discrete droplets can be generated or otherwise provided. Moreover, the plurality of discrete droplets may comprise both unoccupied and occupied droplets.

As described elsewhere herein, in some embodiments of the compositions and methods of the present disclosure, the generated discrete droplets containing a biological sample, a nano-partition containing an enzyme, and optionally, one or more different beads, also contain other reagents. In some embodiments, the other reagents encapsulated or contained in the droplet include lysis and/or un-fixing agents that act to release and/or un-fix the biomolecule contents of the biological sample particle within the droplet. In some embodiments, the lysis and/or un-fixing agents can be contacted with the biological sample suspension concurrently with, or immediately prior to, the introduction of the biological sample particles into the droplet generation junction of the microfluidic system (e.g., junction 210). In some embodiments, the agents are introduced through an additional channel or channels upstream of the channel junction.

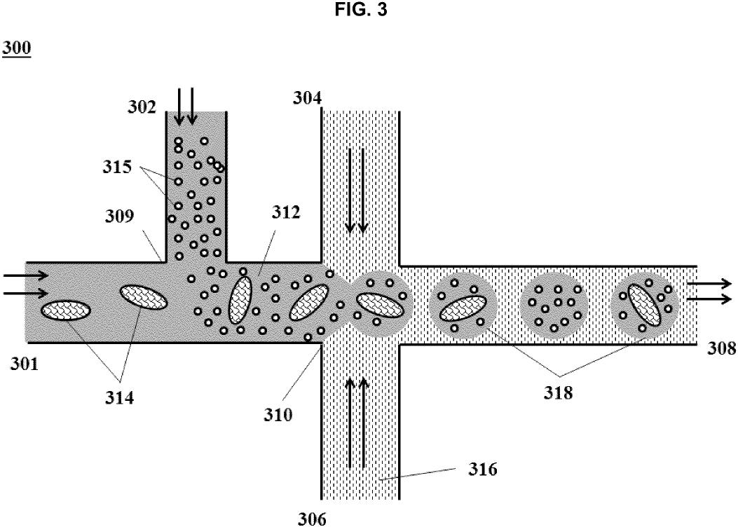

In some embodiments, a biological sample and a nano-partition can be co-partitioned along with the other reagents. FIG. 3 shows an example of a microfluidic channel structure 300 for co-partitioning cells or particles of a biological sample and other reagents, including lysis and/or un-fixing agents. The channel structure 300 can include channel segments 301, 302, 304, 306 and 308. Channel segments 301 and 302 communicate at a first channel junction 309. Channel segments 302, 304, 306, and 308 communicate at a second channel junction 310. In exemplary co-partitioning operation, the channel segment 301 may transport an aqueous fluid 312 that includes a plurality of biological sample cells or particles 314 (e.g., a fixed biological sample) along the channel segment 301 into the second junction 310. As an alternative or in addition to, channel segment 301 may transport a fluid carrying a nano-partition (e.g., a degradable hydrogel matrix with an encapsulated reverse-transcriptase). For example, the channel segment 301 may be connected to a reservoir comprising an aqueous suspension of the biological sample cells or particles 314. Upstream of, and immediately prior to reaching, the second junction 310, the channel segment 301 may meet the channel segment 302 at the first junction 309. The channel segment 302 can transport a plurality of reagents 315 (e.g., lysis or un-fixing agents) in the aqueous fluid 312 along the channel segment 302 into the first junction 309. For example, the channel segment 302 may be connected to a reservoir comprising the reagents 315. After the first junction 309, the aqueous fluid 312 in the channel segment 301 can carry both the biological sample particles 314 and the reagents 315 towards the second junction 310. In some instances, the aqueous fluid 312 in the channel segment 301 can include one or more reagents, which can be the same or different reagents as the reagents 315. A second fluid 316 that is immiscible with the aqueous fluid 312 (e.g., a fluorinated oil) can be delivered to the second junction 310 from each of channel segments 304 and 306. Upon meeting of the aqueous fluid 312 from the channel segment 301 and the second fluid 316 from each of channel segments 304 and 306 at the second channel junction 310, the aqueous fluid 312 is partitioned as discrete droplets 318 in the second fluid 316 and flow away from the second junction 310 along channel segment 308. The channel segment 308 may deliver the discrete droplets 318 to an outlet reservoir fluidly coupled to the channel segment 308, where they may be collected for further analysis.

Discrete droplets generated can include an individual biological sample cell or particle 314 and/or one or more reagents 315, depending on what reagents are included in channel segment 302. The discrete droplet generated may also include a degradable nano-partition and/or a bead (not shown), such as can be added via other channel structures described elsewhere herein. In some instances, a discrete droplet may be unoccupied (e.g., no reagents, no biological particles). Generally, the channel segments described herein may be coupled to any of a variety of different fluid sources or receiving components, including reservoirs, tubing, manifolds, or fluidic components of other systems. As will be appreciated, the microfluidic channel structure 300 may have other geometries. For example, a microfluidic channel structure can have more than two channel junctions. For example, a microfluidic channel structure can have 2, 3, 4, 5 channel segments or more each carrying the same or different types of degradable nano-partitions, beads, reagents, and/or biological sample cells or particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electro-kinetic pumping, vacuum, capillary or gravity flow, or the like.

FIG. 4 shows an example of a microfluidic channel structure for the controlled partitioning of degradable nano-partitions or beads into discrete droplets. A channel structure 400 can include a channel segment 402 communicating at a channel junction 406 (or intersection) with a reservoir 404. The reservoir 404 can be a chamber. Any reference to “reservoir,” as used herein, can also refer to a “chamber.” In operation, an aqueous fluid 408 that includes suspended beads 412 may be transported along the channel segment 402 into the junction 406 to meet a second fluid 410 that is immiscible with the aqueous fluid 408 in the reservoir 404 to create droplets 416, 418 of the aqueous fluid 408 flowing into the reservoir 404. At the junction 406 where the aqueous fluid 408 and the second fluid 410 meet, droplets can form based on factors such as the hydrodynamic forces at the junction 406, flow rates of the two fluids 408, 410, fluid properties, and certain geometric parameters (e.g., w, h₀, α, etc.) of the channel structure 400. A plurality of droplets can be collected in the reservoir 404 by continuously injecting the aqueous fluid 408 from the channel segment 402 through the junction 406.

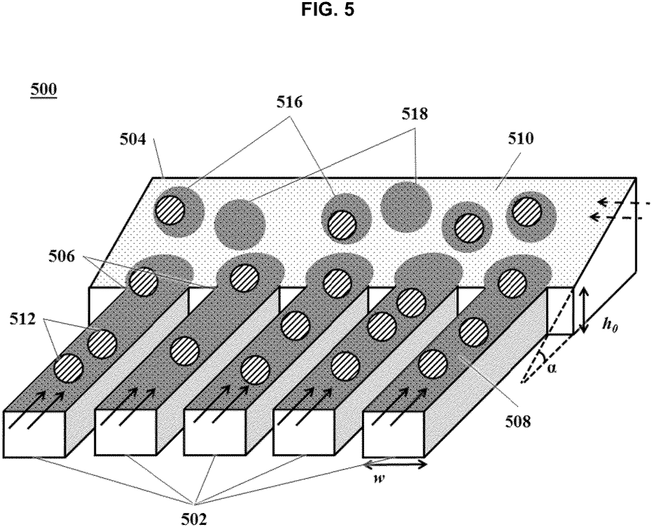

FIG. 5 shows an example of a microfluidic channel structure for increased droplet generation throughput. A microfluidic channel structure 500 can comprise a plurality of channel segments 502 and a reservoir 504. Each of the plurality of channel segments 502 may be in fluid communication with the reservoir 504. The channel structure 500 can comprise a plurality of channel junctions 506 between the plurality of channel segments 502 and the reservoir 504. Each channel junction can be a point of droplet generation. The channel segment 402 from the channel structure 400 in FIG. 4 and any description to the components thereof may correspond to a given channel segment of the plurality of channel segments 502 in channel structure 500 and any description to the corresponding components thereof. The reservoir 404 from the channel structure 400 and any description to the components thereof may correspond to the reservoir 504 from the channel structure 500 and any description to the corresponding components thereof.

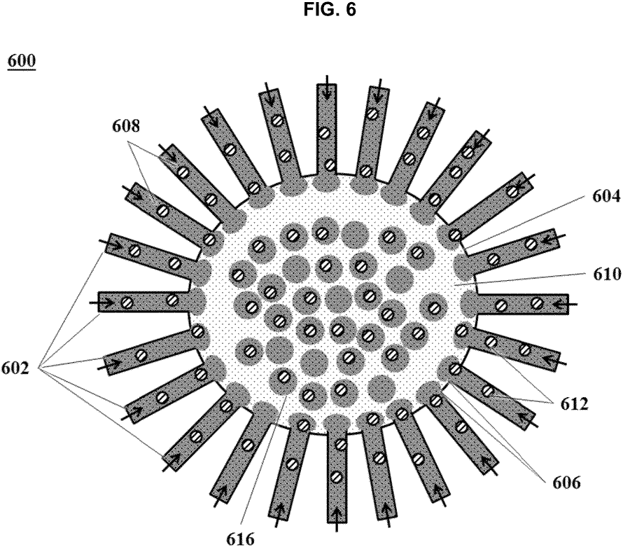

FIG. 6 shows another example of a microfluidic channel structure for increased droplet generation throughput. A microfluidic channel structure 600 can comprise a plurality of channel segments 602 arranged generally circularly around the perimeter of a reservoir 604. Each of the plurality of channel segments 602 may be in fluid communication with the reservoir 604. The channel structure 600 can comprise a plurality of channel junctions 606 between the plurality of channel segments 602 and the reservoir 604. Each channel junction can be a point of droplet generation. The channel segment 402 from the channel structure 400 in FIG. 6 and any description to the components thereof may correspond to a given channel segment of the plurality of channel segments 602 in channel structure 600 and any description to the corresponding components thereof. The reservoir 404 from the channel structure 400 and any description to the components thereof may correspond to the reservoir 604 from the channel structure 600 and any description to the corresponding components thereof. Additional aspects of the microfluidic structures depicted in FIGS. 4-6, including systems and methods implementing the same, are provided in U.S. Pat. Publ. No. 2019/0323088A1, which is incorporated herein by reference in its entirety.

Once a protease, un-fixing agent, and a nano-partition another enzyme are co-partitioned in a droplet containing, the protease and un-fixing agent can begin acting facilitate the release and un-fixing of the biomolecular contents of the biological sample within the droplet. As described elsewhere herein, the un-fixed biomolecular contents released in a droplet remain discrete from the contents of other droplets, thereby allowing for detection and quantitation of the biomolecular analytes of interest present in that distinct biological sample. Furthermore, until the nano-partition is degraded, the enzyme contained within it does not interact with the protease and thus, retains the activity needed to carry out its function for generating biomolecular analytes, such as cDNA.

In some embodiment, a lysis agent can be included in the partition. Examples of lysis agents useful in the compositions and methods of the present disclosure include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents may additionally or alternatively be co-partitioned with the biological sample to cause the release of its contents into the droplet. For example, in some cases, surfactant-based lysis solutions may be used to lyse cells, although these may be less desirable for emulsion based systems where the surfactants can interfere with stable emulsions. In some embodiment, the lysis solutions can include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion based partitioning such as encapsulation of biological particles (e.g., cells, fixed cells, un-fixed cells, organelles, fixed organelles, unfixed organelles, nucleus of a cell, fixed nucleus of a cell, unfixed nucleus of a cell, etc.) that may be in addition to or in place of droplet partitioning, where any pore size of the encapsulate is sufficiently small to retain nucleic acid fragments of a given size, following cellular disruption.

In addition to the lysis and/or un-fixing agents co-partitioned into discrete droplets with the biological sample, it is further contemplated that other assay reagents can also be co-partitioned in the droplet. For example, DNase and RNase inactivating agents or inhibitors, chelating agents, such as EDTA, proteases, such as subtilisin A, proteinase K, Serratia peptidase (i.e., peptidase derived from Serratia sp.), ArcticZymes Proteinase, and other reagents employed in removing or otherwise reducing negative activity or impact of different cell lysate components on subsequent processing of nucleic acids.

As described elsewhere herein, reagents used in subsequent processing of nucleic acids can include nucleic acid processing enzymes, such as a reverse transcriptase, a polymerase, a terminal transferase, a cas enzyme, a restriction enzyme, a USER enzyme, a transposase, and/or a ligase, which are encapsulated in a nano-partition contained within the larger partition. These enzymes can thus be protected from other reagents (such as proteases) until the partition is exposed to an appropriate stimulus that degrades the nano-partition and releases the enzyme(s). In some embodiments, the stimulus may be a chemical reagent, such as DTT, that is co-partitioned in the droplet along with a fixing biological sample, a protease, and an un-fixing agent. The presence of the DTT will commence degrading a nano-partition comprising a hydrogel matrix with cleavable disulfide crosslinks, and eventually release the enzyme contents of the nano-partition into the droplet. In some embodiments, the nano-partition can be degraded by heat stimulus. In such an embodiment, the droplet is exposed to heat at some point in time, typically after the protease and un-fixing agent have had sufficient time to act on the fixed biological sample, and thereby heat inactivate the protease and concurrently cause the release of the second enzyme (e.g., reverse transcriptase) into the droplet to commence it action on the un-fixed nucleic acids.

In another embodiment, it is contemplated that at least two nano-partitions can be partitioned along with a biological sample, wherein the two nano-partitions carry different reagents (e.g., one bead carrying an un-fixing agent, and one bead carrying an enzyme), wherein the contents of the two different nano-partitions are released by non-overlapping stimuli (e.g., a chemical stimulus and a heat stimulus). Such an embodiment can allow the release of the different reagents into the same discrete droplet at different times. For example, a first nano-partition, degraded by heat stimulus, releases an un-fixing agent into the droplet, and then after a set time, a second bead, degraded by a chemical stimulus, releases reverse transcriptase that catalyzes the synthesis of cDNA from the un-fixed mRNA in the partition.

Additional assay reagents that may also be co-partitioned in a nano-partition within a discrete droplet along with a biological sample, can include endonucleases to fragment a biological sample’s DNA, DNA polymerase enzymes and dNTPs used to amplify the biological sample’s nucleic acid fragments and to attach the barcode molecular tags to the amplified fragments. Other nucleic acid processing enzymes that may be co-partitioned in a nano-partition, include, without limitation, a polymerase, a cas enzyme, a restriction enzyme, a USER enzyme, a transposase, a ligase, a DNase, a reverse transcriptase (RT) enzymes, and other enzymes with terminal transferase activity. Additionally, it is contemplated that in some embodiments, co-substrates and reagents used by DNA processing enzymes, such as primers, oligonucleotides, and switch oligonucleotides (also referred to herein as “switch oligos” or “template switching oligonucleotides”) can be encapsulated, embedded or entrapped in the nano-partition along with the enzyme.

Generally, template switching is used to increase the length of cDNA generated in an assay. In some embodiments, template switching can be used to append a predefined nucleic acid sequence to the cDNA. In an example of template switching, cDNA can be generated from reverse transcription of a template, e.g., cellular mRNA, where a reverse transcriptase (RT) with terminal transferase activity can add additional nucleotides, e.g., polyC, to the cDNA in a template independent manner.

Once the contents of a biological sample cell are released into a discrete droplet, the biomolecular components (e.g., macromolecular constituents of biological samples, such as RNA, DNA, or proteins) contained therein may be further processed within the droplet. In accordance with the methods and systems described herein, the biomolecular contents of individual biological samples can be provided with unique barcode identifiers, and upon characterization of the biomolecular components (e.g., in a sequencing assay) they may be attributed as having been derived from the same biological sample. The ability to attribute characteristics to individual biological samples or groups of biological samples is provided by the assignment of a nucleic acid barcode sequence specifically to an individual biological sample or groups of biological samples.

In some aspects, the unique identifiers are barcode molecules provided in the form of nucleic acid molecules (e.g., oligonucleotides). These barcode molecules comprise specific sequences that may be attached to or otherwise associated with the nucleic acid contents of individual biological sample, or to other components of the biological sample, and particularly to fragments of those nucleic acids. In some embodiments, only one nucleic acid barcode sequence is associated with a given discrete droplet, although in some cases, two or more different barcode sequences may be present. The nucleic acid barcode sequences can include from about 6 to about 20 or more nucleotides within the sequence of the nucleic acid molecules (e.g., oligonucleotides). In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

In some embodiments, the nucleic acid barcode molecules can also comprise other functional sequences useful in the processing of the nucleic acids from the biological sample in the droplet. These functional sequences can include, e.g., targeted or random/universal amplification primer sequences for amplifying the nucleic acid molecules from the individual biological samples within the partitions while attaching the associated barcode sequences, sequencing primers or primer recognition sites, hybridization or probing sequences, e.g., for identification of presence of the sequences or for pulling down barcoded nucleic acid molecules, or any of a number of other potential functional sequences.

In some embodiments, large numbers of nucleic acid barcode molecules (e.g., oligonucleotides) are releasably attached to beads, wherein all of the nucleic acid molecules attached to a particular bead will include the same nucleic acid barcode sequence, but where a large number of diverse barcode sequences are represented across the population of beads used. In some embodiments, gel beads (e.g., comprising polyacrylamide polymer matrices), are used as a solid support and delivery vehicle for the nucleic acid molecules into the droplets, as they are capable of carrying large numbers of nucleic acid molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more.

In some embodiments, the nucleic acid barcode molecules are released from a bead contained in a partition upon the application of a particular stimulus. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules form the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological samples and may be degraded for release of the attached nucleic acid molecules through exposure to a reducing agent, such as DTT.

### E. Use in Partition-Based Assays

As disclosed elsewhere herein, the compositions and methods of the present disclose allow biological sample (e.g., formaldehyde-fixed biopsy cells) to be encapsulated in a partition (e.g., a discrete droplet; optionally, as a single cell,or a nuclei from a single cell) together with a first enzyme and second enzyme, wherein the second enzyme is contained by a nano-partition within the partition that prevents it from interacting with the first enzyme. The containment of the second enzyme in a nano-partition facilitates the use of more complex assays that utilize incompatible enzymes at different points in time. Thus, in one embodiment, the present disclosure provides an assay method that comprises the steps of: (a) providing a partition containing a biological sample, a first enzyme, a second enzyme, and a nano-partition, wherein the first and second enzymes catalyze different reactions, and wherein the nano-partition separates the second enzyme from the biological sample and the first enzyme, thereby preventing the first and second enzymes from interacting; (b) wherein the first enzyme catalyzes a reaction with the biological sample; and (c) wherein the second enzyme catalyzes a reaction with the biological sample, or a component thereof. In at least one embodiment, steps (b) and (c) occur simultaneously. In at least one embodiment, step (c) occurs after (b).

In at least one embodiment of this assay method, the first and second enzymes are incompatible; optionally, wherein the first enzyme degrades the second enzyme, and/or reduces the activity of the second enzyme.

In at least one embodiment of this assay method, the reaction of the first enzyme with the biological sample generates a substrate for the reaction of the second enzyme; optionally wherein the generating comprises rendering the substrate accessible to the second enzyme.

In at least one embodiment of this assay method, the reaction of the second enzyme with the biological sample generates analytes. In at least one embodiment, the assay method further comprises a step of detecting the generated analytes.

In at least one embodiment of this assay method, the first enzyme is a protease, (such as alcalase, alkaline proteinase, ArcticZymes Proteinase, bacillopeptidase A, bacillopeptidase B, bioprase, colistinase, esperase, genenase, kazusase, maxatase, pepsin, Serratia peptidase, proteinase K, protease S, savinase, subtilisin A, subtilisin B, subtilisin BL, subtilisin E, subtilisin J, subtilisin S, subtilisin S41, thermoase, and trypsin, or a combination thereof), and the second enzyme is a nucleic acid processing enzyme, such as a reverse transcriptase, a polymerase, a terminal transferase, a cas enzyme, a restriction enzyme, a USER enzyme, a transposase, and/or a ligase.

The ability to use a protease in combination with a nucleic acid processing enzyme (e.g., a reverse transcriptase, a polymerase, a terminal transferase, a cas enzyme, a restriction enzyme, a USER enzyme, a transposase, and/or a ligase) in an assay method carried out in a single partition facilitates assays using fixed biological samples. In such an embodiment of the assay method, the partition further comprises an un-fixing agent along with the protease, and a stimulus (such as heat) can be applied that inactivates the protease as well as degrades the nano-partition to release the second enzyme, which then commences the nucleic acid processing portion of the assay (e.g., cDNA synthesis from un-fixed mRNA).

The ability to un-fix a previously fixed biological sample in a partition allows the cellular analytes of the sample to be assayed as if they were obtained from a fresh sample. Thus, it is contemplated that in some embodiments of the methods a fresh biological sample is immediately fixed e.g., with formaldehyde, and then stored for a period of time before it is partitioned with protease, an un-fixing agent, a nano-partition containing a nucleic acid processing enzyme (e.g., a reverse transcriptase, a polymerase, a terminal transferase, a cas enzyme, a restriction enzyme, a USER enzyme, a transposase, and/or a ligase), and other materials such as unique nucleic acid barcode molecule and assay reagents. Further the methods of the present disclosure can be carried out wherein the amount of time prior to generating the partition when the biological sample is fixed is at least 1 hour, at least 2 hours, at least 6 hours, at least 12 hours, at least 24 hours, at least 1 week, at least 1 month, at least 6 months, or longer.

Types of cellular analytes that can be assayed using with the compositions and methods of the present disclosure include, without limitation, intracellular and partially intracellular analytes, including a protein, a metabolite, a metabolic byproduct, an antibody or antibody fragment, an enzyme, an antigen, a carbohydrate, a lipid, a macromolecule, or a combination thereof (e.g., proteoglycan) or other biomolecule. The cellular analyte may be a nucleic acid molecule, such as a deoxyribonucleic acid (DNA) molecule (e.g., genomic DNA) or a ribonucleic acid (RNA) molecule (e.g., messenger RNA (mRNA), ribosomal RNA (rRNA) or transfer RNA (tRNA)).

In at least one embodiment of the present disclosure, the cellular analyte detected from a biological sample can be an RNA transcript, such as in a gene expression profiling assay. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA.

In at least one embodiment, the cellular analyte detected is associated with an intermediary entity, wherein the intermediary entity is analyzed to provide information about the cellular analyte and/or the intermediary entity itself. For instance, an intermediary entity (e.g., an antibody) may be bound to a partially intracellular analyte (e.g., a cell surface receptor), where the intermediary entity is processed to provide information about the intermediary entity, the partially intracellular analyte, or both. In at least one embodiment, the intermediary entity comprises an identifier of the biological sample, such as a barcode oligonucleotide, as further described herein.

The present disclosure also provides an assay method that comprises the steps of: (a) generating a partition containing a biological sample, a first enzyme, a second composition enzyme, assay reagents, and a nano-partition, wherein the nano-partition separates the second enzyme from the biological sample and the first enzyme, thereby preventing the first and second enzymes from interacting; (b) providing a stimulus that degrades the nano-partition; and (c) detecting analytes from the reaction of the biological sample, the assay reagents and the second enzyme type.

The assay methods of the present disclosure can by carried out using fixed biological samples. Optionally, the steps of the assay method can further comprise a step fixing the biological sample prior to generating the partition. For example, the present disclosure also provides an assay method that comprises the steps of: (a) fixing a biological sample; (b) generating a partition encapsulating the fixed biological sample, an un-fixing agent, a protease (as first enzyme), a nano-partition containing a nucleic acid processing enzyme (as second enzyme), and assay reagents; (c) inactivating the protease prior to, or concurrent with, degrading the nano-partition with a stimulus; and (d) detecting analytes from the reaction of the second enzyme, the assay reagents and the un-fixed biological sample.

A wide range of partition-based materials, methods, assays, and systems suitable for use in the embodiments of the present disclosure are known in the art, and described in U.S. Pat. Nos. 9,694,361, 10,357,771, 10,273,541, and 10,011,872, and U.S. Pat. Publ. Nos. 2018/0105808A1, 2019/0367982A1, and 2019/0338353A1, each of which is hereby incorporated herein by reference in its entirety. It is contemplated that any assay that can be carried out using a fresh biological sample, such as a single cell, an organelle of a single cell, and/or a nucleus of a single cell, encapsulated in a droplet with a bead carrying a barcode, can also be carried out using a partition containing a biological sample, a first enzyme, a nano-partition containing a second enzyme, and the methods of the present disclosure.

Exemplary assays include single-cell transcription profiling, single-cell sequence analysis, immune profiling of individual T and B cells, single-cell chromatin accessibility analysis (e.g., ATAC seq analysis). These exemplary assays can be carried out using commercially available systems for encapsulating biological samples, gel beads, barcodes, and/or other compounds/materials in droplets, such as The Chromium System (10X Genomics, Pleasanton, CA, USA).

As described elsewhere herein, in some embodiments of the assay methods, the discrete droplet further comprises one or more beads. In some embodiments, the bead(s) can contain the assay reagents and/or the un-fixing agent. In some embodiments, a barcode is carried by or contained in a bead. Compositions, methods and systems for sample preparation, amplification, and sequencing of biomolecules from single cells encapsulated with barcodes in droplets are provided in e.g., U.S. Pat. Publ. No. 2018/0216162A1, which is hereby incorporated by reference herein.

Assay reagents can include those used to perform one or more additional chemical or biochemical operations on a biological sample encapsulated in a droplet. Accordingly, assay reagents useful in the assay method include any reagents useful in performing a reaction such as nucleic acid modification (e.g., ligation, digestion, methylation, random mutagenesis, bisulfite conversion, uracil hydrolysis, nucleic acid repair, capping, or decapping), nucleic acid amplification (e.g., isothermal amplification or PCR), nucleic acid insertion or cleavage (e.g., via CRISPR/Cas9-mediated or transposon-mediated insertion or cleavage), and/or reverse transcription. Additionally, useful assay reagents can include those that allow the preparation of a target sequence or sequencing reads that are specific to the macromolecular constituents of interest at a higher rate than to non-target sequence specific reads.

In addition, the present invention provides compositions and systems related to the analysis of fixed biological samples. In one embodiment, the present invention provides a composition comprising a plurality of partitions, wherein a subset of said plurality of partitions comprises fixed cells and an un-fixing agent. In one other embodiment, the subset of partitions further comprises a protease. In another embodiment, a partition of the plurality of partitions comprises a fixed cell and an un-fixing agent. In certain embodiments, the fixed cell is a single fixed cell. In other embodiments the present invention provides a composition comprising a partition, wherein the partition comprises a fixed cell and an un-fixing agent, as described herein. The partition may be a droplet or a well. In another embodiment, the partition further comprises a protease. The partition or partitions described herein may further comprise one or more of the following: a reverse transcriptase (RT), a bead, and reagents for a nucleic acid extension reaction. In an additional embodiment, the compositions of the present invention have or are provided at a temperature other than ambient temperature or non-ambient temperature. In one embodiment, the temperature is below ambient temperature or above ambient temperature. As described elsewhere herein, partitioning approaches may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions. For example, an occupied partition according the present invention comprises a fixed cell and an un-fixing agent.

In another aspect, the present invention concerns methods and compositions for the partitioning of a plurality of fixed cells into individual partitions. In some cases, about 10, about 20, about 30, about 40, about 50, about 60, about 70, about 80, about 90, about 100, about 200, about 300, about 400, about 500, about 600, about 700, about 800, about 900, about 1000, about 2000, about 3000, about 4000, about 5000, about 6000, about 7000, about 8000, about 9000, about 10,000, about 15,000, about 20,000, about 25,000, about 30,000, about 35,000, about 40,000, about 50,000, about 60,000, about 70,000, about 80,000, about 90,000 or about 100,000 fixed cells may be partitioned into individual partitions. In some instances, the method further comprises partitioning about 50 to about 20,000 fixed cells with each of a plurality of supports comprising the adaptor comprising the barcode sequence, wherein the barcode sequence is unique among each of the plurality of supports.

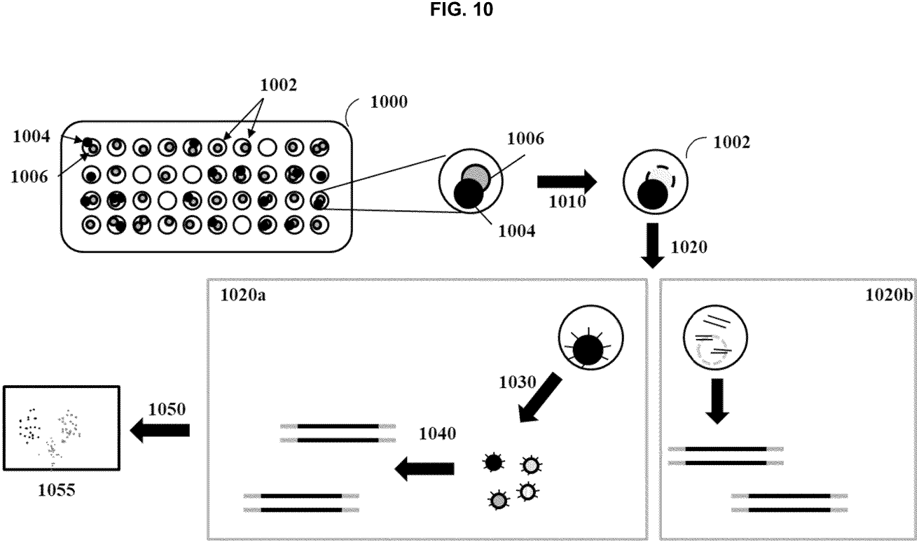

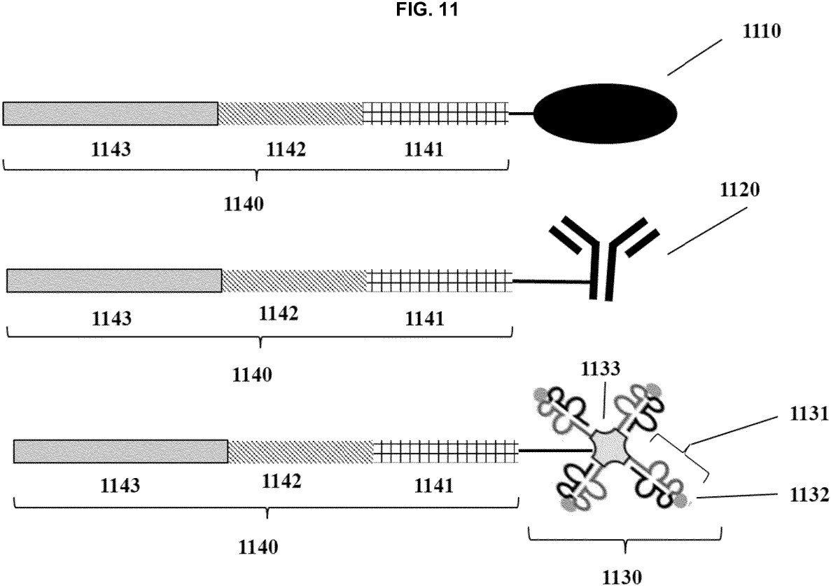

FIG. 9 schematically illustrates an example of a microwell array. The array can be contained within a substrate 900. The substrate 900 comprises a plurality of wells 902. The wells 902 may be of any size or shape, and the spacing between the wells, the number of wells per substrate, as well as the density of the wells on the substrate 900 can be modified, depending on the particular application. In one such example application, a sample molecule 906, which may comprise a cell (e.g., a fixed cell or an un-fixed cell) or cellular components (e.g., nucleic acid molecules) is co-partitioned with a bead 904, which may comprise a nucleic acid barcode molecule coupled thereto. The wells 902 may be loaded using gravity or other loading technique (e.g., centrifugation, liquid handler, acoustic loading, optoelectronic, etc.). In some instances, at least one of the wells 902 contains a single sample molecule 906 (e.g., cell) and a single bead 904.

Reagents may be loaded into a well either sequentially or concurrently. In some cases, reagents are introduced to the device either before or after a particular operation. In some cases, reagents (which may be provided, in certain instances, in droplets or beads) are introduced sequentially such that different reactions or operations occur at different steps. The reagents (or droplets or beads) may also be loaded at operations interspersed with a reaction or operation step. For example, droplets or beads comprising reagents for fragmenting polynucleotides (e.g., restriction enzymes) and/or other enzymes (e.g., transposases, ligases, polymerases, etc.) may be loaded into the well or plurality of wells, followed by loading of droplets or beads comprising reagents for attaching nucleic acid barcode molecules to a sample nucleic acid molecule. Reagents may be provided concurrently or sequentially with a sample, such as a cell (e.g., a fixed cell or an un-fixed cell) or cellular components (e.g., organelles, proteins, nucleic acid molecules, carbohydrates, lipids, etc.). Accordingly, use of wells may be useful in performing multi-step operations or reactions.

As described elsewhere herein, the nucleic acid barcode molecules and other reagents may be contained within a bead or droplet. These beads or droplets may be loaded into a partition (e.g., a microwell) before, after, or concurrently with the loading of a cell (e.g., a fixed cell or an un-fixed cell), such that each cell is contacted with a different bead or droplet. This technique may be used to attach a unique nucleic acid barcode molecule to nucleic acid molecules obtained from each cell (e.g., a fixed cell or an un-fixed cell). Alternatively or in addition to, the sample nucleic acid molecules may be attached to a support. For instance, the partition (e.g., microwell) may comprise a bead which has coupled thereto a plurality of nucleic acid barcode molecules. The sample nucleic acid molecules, or derivatives thereof, may couple or attach to the nucleic acid barcode molecules on the support. The resulting barcoded nucleic acid molecules may then be removed from the partition, and in some instances, pooled and sequenced. In such cases, the nucleic acid barcode sequences may be used to trace the origin of the sample nucleic acid molecule. For example, polynucleotides with identical barcodes may be determined to originate from the same cell or partition, while polynucleotides with different barcodes may be determined to originate from different cells or partitions.

The samples or reagents may be loaded in the wells or microwells using a variety of approaches. The samples (e.g., a cell or cellular component) or reagents (as described herein) may be loaded into the well or microwell using an external force, e.g., gravitational force, electrical force, magnetic force, or using mechanisms to drive the sample or reagents into the well, e.g., via pressure-driven flow, centrifugation, optoelectronics, acoustic loading, electrokinetic pumping, vacuum, capillary flow, etc. In certain cases, a fluid handling system may be used to load the samples or reagents into the well. The loading of the samples or reagents may follow a Poissonian distribution or a non-Poissonian distribution, e.g., super Poisson or sub-Poisson. The geometry, spacing between wells, density, and size of the microwells may be modified to accommodate a useful sample or reagent distribution; for instance, the size and spacing of the microwells may be adjusted such that the sample or reagents may be distributed in a super-Poissonian fashion.

In one particular non-limiting example, the microwell array or plate comprises pairs of microwells, in which each pair of microwells is configured to hold a droplet (e.g., comprising a single cell, e.g., a single fixed cell or a single un-fixed cell) and a single bead (such as those described herein, which may, in some instances, also be provided or encapsulated in a droplet). The droplet and the bead (or droplet containing the bead) may be loaded simultaneously or sequentially, and the droplet and the bead may be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets comprising different reagents and/or samples, which are merged upon contact or upon application of a stimulus. In such instances, the droplet of one microwell of the pair can comprise reagents that may react with an agent in the droplet of the other microwell of the pair. For instance, one droplet can comprise reagents that are configured to release the nucleic acid barcode molecules of a bead contained in another droplet, located in the adjacent microwell. Upon merging of the droplets, the nucleic acid barcode molecules may be released from the bead into the partition (e.g., the microwell or microwell pair that are in contact), and further processing may be performed (e.g., barcoding, nucleic acid reactions, etc.). In cases where cells, e.g., fixed cells or un-fixed cells are loaded in the microwells, one of the droplets may comprise reagents for further processing, e.g., lysis reagents for lysing the cell, upon droplet merging.

A droplet may be partitioned into a well. The droplets may be selected or subjected to pre-processing prior to loading into a well. For instance, the droplets may comprise cells, e.g., fixed cells or un-fixed cells, and only certain droplets, such as those containing a single cell (or at least one cell), may be selected for use in loading of the wells. Such a pre-selection process may be useful in efficient loading of single cells, such as to obtain a non-Poissonian distribution, or to pre-filter cells for a selected characteristic prior to further partitioning in the wells. Additionally, the technique may be useful in obtaining or preventing cell doublet or multiplet formation prior to or during loading of the microwell.

In some instances, the wells can comprise nucleic acid barcode molecules attached thereto. The nucleic acid barcode molecules may be attached to a surface of the well (e.g., a wall of the well). The nucleic acid barcode molecule (e.g., a partition barcode sequence) of one well may differ from the nucleic acid barcode molecule of another well, which can permit identification of the contents contained with a single partition or well. In some cases, the nucleic acid barcode molecule can comprise a spatial barcode sequence that can identify a spatial coordinate of a well, such as within the well array or well plate. In some cases, the nucleic acid barcode molecule can comprise a unique molecular identifier for individual molecule identification. In some instances, the nucleic acid barcode molecules may be configured to attach to or capture a nucleic acid molecule within a sample or cell (e.g., a fixed cell or an un-fixed cell) distributed in the well. For example, the nucleic acid barcode molecules may comprise a capture sequence that may be used to capture or hybridize to a nucleic acid molecule (e.g., RNA, DNA) within the sample. In some instances, the nucleic acid barcode molecules may be releasable from the microwell. For instance, the nucleic acid barcode molecules may comprise a chemical cross-linker which may be cleaved upon application of a stimulus (e.g., photo-, magnetic, chemical, biological, stimulus). The released nucleic acid barcode molecules, which may be hybridized or configured to hybridize to a sample nucleic acid molecule, may be collected and pooled for further processing, which can include nucleic acid processing (e.g., amplification, extension, reverse transcription, etc.) and/or characterization (e.g., sequencing). In such cases, the unique partition barcode sequences may be used to identify the cell or partition from which a nucleic acid molecule originated.

Characterization of samples within a well may be performed. Such characterization can include, in non-limiting examples, imaging of the sample (e.g., cell or cellular components) or derivatives thereof. Characterization techniques such as microscopy or imaging may be useful in measuring sample profiles in fixed spatial locations. For instance, when cells (e.g., fixed cells or un-fixed cells) are partitioned, optionally with beads, imaging of each microwell and the contents contained therein may provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, cell-cell interactions (when two or more cells are co-partitioned). Alternatively or in addition to, imaging may be used to characterize a quantity of amplification products in the well.

In operation, a well may be loaded with a sample and reagents, simultaneously or sequentially. When cells (e.g., fixed cells or un-fixed cells) are loaded, the well may be subjected to washing, e.g., to remove excess cells from the well, microwell array, or plate. Similarly, washing may be performed to remove excess beads or other reagents from the well, microwell array, or plate. In addition, the cells may be lysed in the individual partitions to release the intracellular components or cellular analytes. Alternatively, the cells may be fixed or permeabilized in the individual partitions. The intracellular components or cellular analytes may couple to a support, e.g., on a surface of the microwell, on a solid support (e.g., bead), or they may be collected for further downstream processing. For instance, after cell lysis, the intracellular components or cellular analytes may be transferred to individual droplets or other partitions for barcoding. Alternatively, or in addition to, the intracellular components or cellular analytes (e.g., nucleic acid molecules) may couple to a bead comprising a nucleic acid barcode molecule; subsequently, the bead may be collected and further processed, e.g., subjected to nucleic acid reaction such as reverse transcription, amplification, or extension, and the nucleic acid molecules thereon may be further characterized, e.g., via sequencing. Alternatively, or in addition to, the intracellular components or cellular analytes may be barcoded in the well (e.g., using a bead comprising nucleic acid barcode molecules that are releasable or on a surface of the microwell comprising nucleic acid barcode molecules). The barcoded nucleic acid molecules or analytes may be further processed in the well, or the barcoded nucleic acid molecules or analytes may be collected from the individual partitions and subjected to further processing outside the partition. Further processing can include nucleic acid processing (e.g., performing an amplification, extension) or characterization (e.g., fluorescence monitoring of amplified molecules, sequencing). At any convenient or useful step, the well (or microwell array or plate) may be sealed (e.g., using an oil, membrane, wax, etc.), which enables storage of the assay or selective introduction of additional reagents.

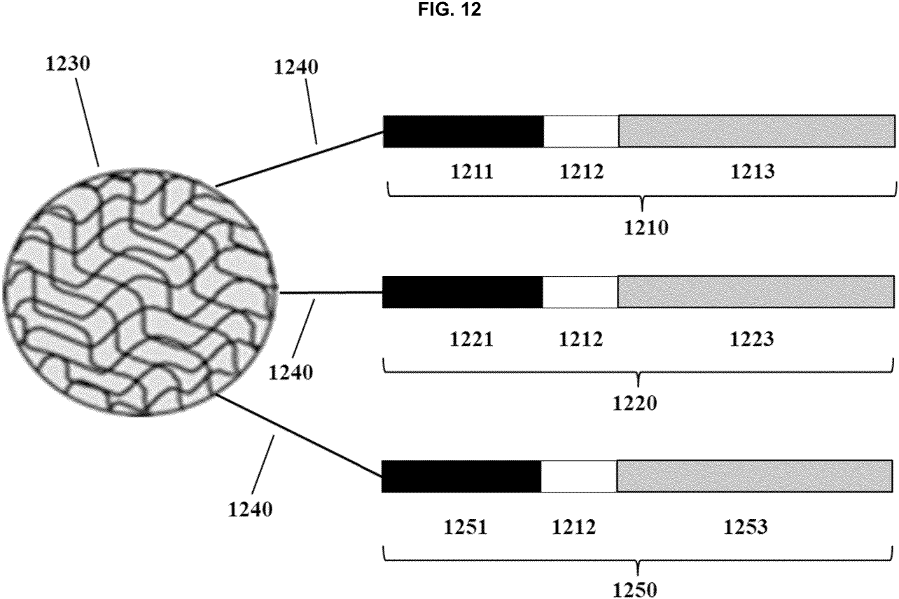

FIG. 10 schematically shows an example workflow for processing nucleic acid molecules within a sample. A substrate 1000 comprising a plurality of microwells 1002 may be provided. A sample 1006 which may comprise a cell (e.g., a fixed cell or an un-fixed cell), cellular components or analytes (e.g., proteins and/or nucleic acid molecules) can be co-partitioned, in a plurality of microwells 1002, with a plurality of beads 1004 comprising nucleic acid barcode molecules. During process 1010, the sample 1006 may be processed within the partition. For instance, the cell may be subjected to conditions sufficient to lyse the cells (e.g., fixed cells or un-fixed cells) and release the analytes contained therein. In process 1020, the bead 1004 may be further processed. By way of example, processes 1020a and 1020b schematically illustrate different workflows, depending on the properties of the bead 1004.

In 1020a, the bead comprises nucleic acid barcode molecules that are attached thereto, and sample nucleic acid molecules (e.g., RNA, DNA) may attach, e.g., via hybridization of ligation, to the nucleic acid barcode molecules. Such attachment may occur on the bead. In process 1030, the beads 1004 from multiple wells 1002 may be collected and pooled. Further processing may be performed in process 1040. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 1050, further characterization, such as sequencing may be performed to generate sequencing reads. The sequencing reads may yield information on individual cells or populations of cells (e.g., fixed cells or un-fixed cells), which may be represented visually or graphically, e.g., in a plot 1055.

In 1020b, the bead comprises nucleic acid barcode molecules that are releasably attached thereto, as described below. The bead may degrade or otherwise release the nucleic acid barcode molecules into the well 1002; the nucleic acid barcode molecules may then be used to barcode nucleic acid molecules within the well 1002. Further processing may be performed either inside the partition or outside the partition. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 1050, further characterization, such as sequencing may be performed to generate sequencing reads. The sequencing reads may yield information on individual cells or populations of cells (e.g., fixed cells or un-fixed cells), which may be represented visually or graphically, e.g., in a plot 1055

In 1020b, the bead comprises nucleic acid barcode molecules that are releasably attached thereto, as described below. The bead may degrade or otherwise release the nucleic acid barcode molecules into the well 1002; the nucleic acid barcode molecules may then be used to barcode nucleic acid molecules within the well 1002. Further processing may be performed either inside the partition or outside the partition. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 1050, further characterization, such as sequencing may be performed to generate sequencing reads. The sequencing reads may yield information on individual cells or populations of cells (e.g., fixed cells or un-fixed cells), which may be represented visually or graphically, e.g., in a plot 1055.

### F. Additional Methods

The present disclosure provides methods and systems for multiplexing, and otherwise increasing throughput of samples (e.g., cells, fixed cells or un-fixed cells) for analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labelling agents capable of binding to or otherwise coupling to one or more cells (e.g., cells, fixed cells or un-fixed cells) or cell features may be used to characterize cells and/or cell features. In some instances, cell features include cell surface features. Cell surface features may include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof. A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence (e.g., a reporter sequence) that permits identification of the labelling agent. For example, a labelling agent that is specific to one type of cell feature (e.g., a first cell surface feature) may have a first reporter oligonucleotide coupled thereto, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. 10,550,429; U.S. Pat. Pub. 20190177800; and U.S. Pat. Pub. 20190367969, each of which is herein entirely incorporated by reference for all purposes.

In a particular example, a library of potential cell feature labelling agents may be provided, where the respective cell feature labelling agents are associated with nucleic acid reporter molecules, such that a different reporter oligonucleotide sequence is associated with each labelling agent capable of binding to a specific cell feature. In other aspects, different members of the library may be characterized by the presence of a different oligonucleotide sequence label. For example, an antibody capable of binding to a first protein may have associated with it a first reporter oligonucleotide sequence, while an antibody capable of binding to a second protein may have a different reporter oligonucleotide sequence associated with it. The presence of the particular oligonucleotide sequence may be indicative of the presence of a particular antibody or cell feature which may be recognized or bound by the particular antibody.

For workflows comprising the use of fixation agents and/or un-fixing agents, labelling agents may be used to label samples (e.g., cells, fixed cells or un-fixed cells) at different points in time. In one embodiment, a plurality of cells is labeled prior to treatment with a fixation agent and/or after treatment with a fixation agent. In another embodiment, a plurality of fixed cells is labeled prior to treatment with an un-fixing agent and/or after treatment with an un-fixing agent. In one additional embodiment, a plurality of un-fixed cells is labeled prior to partitioning into partitions (e.g., wells or droplets) for further processing. In another embodiment, the methods, compositions, systems, and kits described herein provide labeled cells, labeled fixed cells or labeled un-fixed cells.

Labelling agents capable of binding to or otherwise coupling to one or more cells may be used to characterize a cell as belonging to a particular set of cells. For example, labeling agents may be used to label a sample of cells or a group of cells. In this way, a group of cells may be labeled as different from another group of cells. In an example, a first group of cells may originate from a first sample and a second group of cells may originate from a second sample. Labelling agents may allow the first group and second group to have a different labeling agent (or reporter oligonucleotide associated with the labeling agent). This may, for example, facilitate multiplexing, where cells of the first group and cells of the second group may be labeled separately and then pooled together for downstream analysis. The downstream detection of a label may indicate analytes as belonging to a particular group.

For example, a reporter oligonucleotide may be linked to an antibody or an epitope binding fragment thereof, and labeling a cell may comprise subjecting the antibody-linked barcode molecule or the epitope binding fragment-linked barcode molecule to conditions suitable for binding the antibody to a molecule present on a surface of the cell. The binding affinity between the antibody or the epitope binding fragment thereof and the molecules present on the surface may be within a desired range to ensure that the antibody or the epitope binding fragment thereof remains bound to the molecule. For example, the binding affinity may be within a desired range to ensure that the antibody or the epitope binding fragment thereof remains bound to the molecule during various sample processing steps, such as partitioning and/or nucleic acid amplification or extension. A dissociation constant (Kd) between the antibody or an epitope binding fragment thereof and the molecule to which it binds may be less than about 100 µM, 90 µM, 80 µM, 70 µM, 60 µM, 50 µM, 40 µM, 30 µM, 20 µM, 10 µM, 9 µM, 8 µM, 7 µM, 6 µM, 5 µM, 4 µM, 3 µM, 2 µM, 1 µM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 200 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, 10 pM, 9 pM, 8 pM, 7 pM, 6 pM, 5 pM, 4 pM, 3 pM, 2 pM, or 1 pM. For example, the dissociation constant may be less than about 10 µM,

In another example, a reporter oligonucleotide may be coupled to a cell-penetrating peptide (CPP), and labeling cells may comprise delivering the CPP coupled reporter oligonucleotide into an analyte carrier. Labeling analyte carriers may comprise delivering the CPP conjugated oligonucleotide into a cell and/or cell bead by the cell-penetrating peptide. A CPP that can be used in the methods provided herein can comprise at least one non-functional cysteine residue, which may be either free or derivatized to form a disulfide link with an oligonucleotide that has been modified for such linkage. Non-limiting examples of CPPs that can be used in embodiments herein include penetratin, transportan, plsl, TAT(48-60), pVEC, MTS, and MAP. Cell-penetrating peptides useful in the methods provided herein can have the capability of inducing cell penetration for at least about 30%, 40%, 50%, 60%, 70%, 80%, 90%, 95%, 96%, 97%, 98%, 99%, or 100% of cells of a cell population. The CPP may be an arginine-rich peptide transporter. The CPP may be Penetratin or the Tat peptide. In another example, a reporter oligonucleotide may be coupled to a fluorophore or dye, and labeling cells may comprise subjecting the fluorophore-linked barcode molecule to conditions suitable for binding the fluorophore to the surface of the cell. In some instances, fluorophores can interact strongly with lipid bilayers and labeling cells may comprise subjecting the fluorophore-linked barcode molecule to conditions such that the fluorophore binds to or is inserted into a membrane of the cell. In some cases, the fluorophore is a water-soluble, organic fluorophore. In some instances, the fluorophore is Alexa 532 maleimide, tetramethylrhodamine-5-maleimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 635P azide, Atto 647N maleimide, Atto 647 SE, or Sulfo-Cy5 maleimide. See, e.g., Hughes L D, et al. PLoS One. 2014 Feb. 4; 9(2):e87649, which is hereby incorporated by reference in its entirety for all purposes, for a description of organic fluorophores.

A reporter oligonucleotide may be coupled to a lipophilic molecule, and labeling cells may comprise delivering the nucleic acid barcode molecule to a membrane of a cell or a nuclear membrane by the lipophilic molecule. Lipophilic molecules can associate with and/or insert into lipid membranes such as cell membranes and nuclear membranes. In some cases, the insertion can be reversible. In some cases, the association between the lipophilic molecule and the cell or nuclear membrane may be such that the membrane retains the lipophilic molecule (e.g., and associated components, such as nucleic acid barcode molecules, thereof) during subsequent processing (e.g., partitioning, cell permeabilization, amplification, pooling, etc.). The reporter nucleotide may enter into the intracellular space and/or a cell nucleus. In one embodiment, a reporter oligonucleotide coupled to a lipophilic molecule will remain associated with and/or inserted into lipid membrane (as described herein) via the lipophilic molecule until lysis of the cell occurs, e.g., inside a partition.

A reporter oligonucleotide may be part of a nucleic acid molecule comprising any number of functional sequences, as described elsewhere herein, such as a target capture sequence, a random primer sequence, and the like, and coupled to another nucleic acid molecule that is, or is derived from, the analyte.

Prior to partitioning, the cells may be incubated with the library of labelling agents, that may be labelling agents to a broad panel of different cell features, e.g., receptors, proteins, etc., and which include their associated reporter oligonucleotides. Unbound labelling agents may be washed from the cells, and the cells may then be co-partitioned (e.g., into droplets or wells) along with partition-specific barcode oligonucleotides (e.g., attached to a support, such as a bead or gel bead) as described elsewhere herein. As a result, the partitions may include the cell or cells, as well as the bound labelling agents and their known, associated reporter oligonucleotides.

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide. For example, the first plurality of the labeling agent and second plurality of the labeling agent may interact with different cells, cell populations or samples, allowing a particular report oligonucleotide to indicate a particular cell population (or cell or sample) and cell feature. In this way, different samples or groups can be independently processed and subsequently combined together for pooled analysis (e.g., partition-based barcoding as described elsewhere herein). See, e.g., U.S. Pat. Pub. 20190323088, which is hereby entirely incorporated by reference for all purposes.

As described elsewhere herein, libraries of labelling agents may be associated with a particular cell feature as well as be used to identify analytes as originating from a particular cell population, or sample. Cell populations may be incubated with a plurality of libraries such that a cell or cells comprise multiple labelling agents. For example, a cell may comprise coupled thereto a lipophilic labeling agent and an antibody. The lipophilic labeling agent may indicate that the cell is a member of a particular cell sample, whereas the antibody may indicate that the cell comprises a particular analyte. In this manner, the reporter oligonucleotides and labelling agents may allow multi-analyte, multiplexed analyses to be performed.

In some instances, these reporter oligonucleotides may comprise nucleic acid barcode sequences that permit identification of the labelling agent which the reporter oligonucleotide is coupled to. The use of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry such as a Methyltetrazine-PEG5-NHS Ester reaction, a TCO-PEG4-NHS Ester reaction, or the like, may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to an oligonucleotide that is complementary to a sequence of the reporter oligonucleotide, and the oligonucleotide may be allowed to hybridize to the reporter oligonucleotide.

FIG. 11 describes exemplary labelling agents (1110, 1120, 1130) comprising reporter oligonucleotides (1140) attached thereto. Labelling agent 1110 (e.g., any of the labelling agents described herein) is attached (either directly, e.g., covalently attached, or indirectly) to reporter oligonucleotide 1140. Reporter oligonucleotide 1140 may comprise barcode sequence 1142 that identifies labelling agent 1110. Reporter oligonucleotide 1140 may also comprise one or more functional sequences 1143 that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, or a sequencing primer or primer biding sequence (such as an R1, R2, or partial R1 or R2 sequence).

Referring to FIG. 11, in some instances, reporter oligonucleotide 1140 conjugated to a labelling agent (e.g., 1110, 1120, 1130) comprises a primer sequence 1141, a barcode sequence 1142 that identifies the labelling agent (e.g., 1110, 1120, 1130), and functional sequence 1143. Functional sequence 1143 may be configured to hybridize to a complementary sequence, such as a complementary sequence present on a nucleic acid barcode molecule 1190 (not shown), such as those described elsewhere herein. In some instances, nucleic acid barcode molecule 1190 is attached to a support (e.g., a bead, such as a gel bead), such as those described elsewhere herein. For example, nucleic acid barcode molecule 1190 may be attached to the support via a releasable linkage (e.g., comprising a labile bond), such as those described elsewhere herein. In some instances, reporter oligonucleotide 1140 comprises one or more additional functional sequences, such as those described above.

In some instances, the labelling agent 1110 is a protein or polypeptide (e.g., an antigen or prospective antigen) comprising reporter oligonucleotide 1140. Reporter oligonucleotide 1140 comprises barcode sequence 1142 that identifies polypeptide 1110 and can be used to infer the presence of an analyte, e.g., a binding partner of polypeptide 1110 (i.e., a molecule or compound to which polypeptide 1110 can bind). In some instances, the labelling agent 1110 is a lipophilic moiety (e.g., cholesterol) comprising reporter oligonucleotide 1140, where the lipophilic moiety is selected such that labelling agent 1110 integrates into a membrane of a cell or nucleus. Reporter oligonucleotide 1140 comprises barcode sequence 1142 that identifies lipophilic moiety 1110 which in some instances is used to tag cells (e.g., groups of cells, cell samples, etc.) and may be used for multiplex analyses as described elsewhere herein. In some instances, the labelling agent is an antibody 1120 (or an epitope binding fragment thereof) comprising reporter oligonucleotide 1140. Reporter oligonucleotide 1140 comprises barcode sequence 1142 that identifies antibody 1120 and can be used to infer the presence of, e.g., a target of antibody 1120 (i.e., a molecule or compound to which antibody 1120 binds). In other embodiments, labelling agent 1130 comprises an MHC molecule 1131 comprising peptide 1132 and reporter oligonucleotide 1140 that identifies peptide 1132. In some instances, the MHC molecule is coupled to a support 1133. In some instances, support 1133 may be a polypeptide, such as streptavidin, or a polysaccharide, such as dextran. In some instances, reporter oligonucleotide 1140 may be directly or indirectly coupled to MHC labelling agent 1130 in any suitable manner. For example, reporter oligonucleotide 1140 may be coupled to MHC molecule 1131, support 1133, or peptide 1132. In some embodiments, labelling agent 1130 comprises a plurality of MHC molecules, (e.g., is an MHC multimer, which may be coupled to a support (e.g., 1133)). There are many possible configurations of Class I and/or Class II MHC multimers that can be utilized with the compositions, methods, and systems disclosed herein, e.g., MHC tetramers, MHC pentamers (MHC assembled via a coiled-coil domain, e.g., Pro5® MHC Class I Pentamers, (Prolmmune, Ltd.), MHC octamers, MHC dodecamers, MHC decorated dextran molecules (e.g., MHC Dextramer® (Immudex)), etc. For a description of exemplary labelling agents, including antibody and MHC-based labelling agents, reporter oligonucleotides, and methods of use, see, e.g., U.S. Pat. 10,550,429 and U.S. Pat. Pub. 20190367969, each of which is herein entirely incorporated by reference for all purposes.

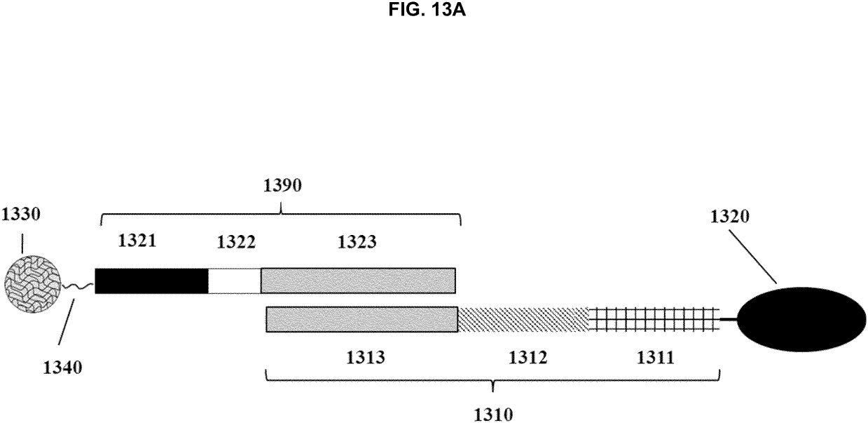

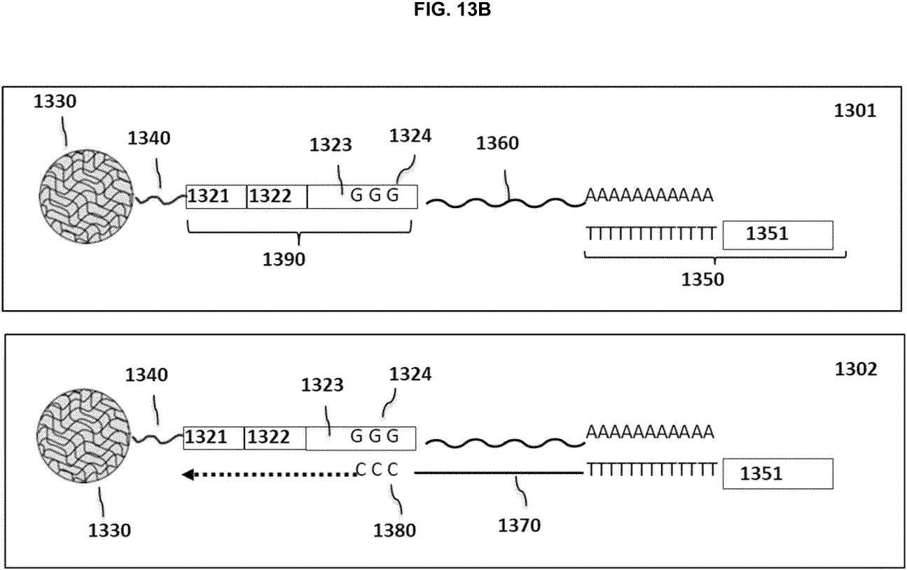

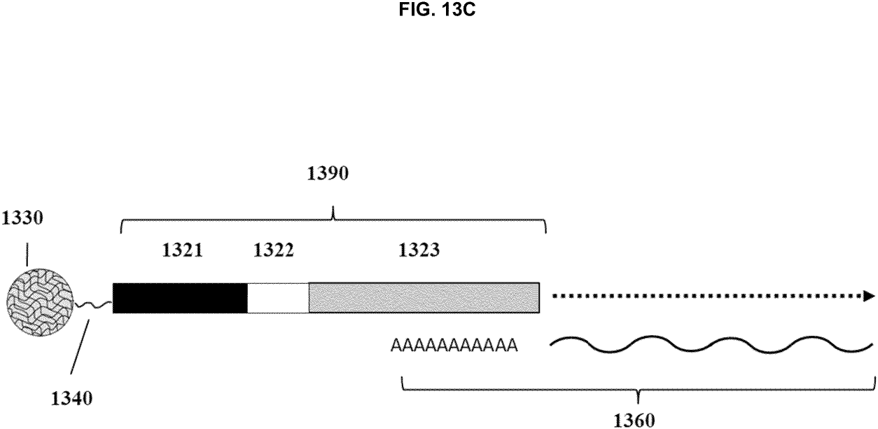

FIG. 12 illustrates another example of a barcode carrying bead. In some embodiments, analysis of multiple analytes (e.g., RNA and one or more analytes using labelling agents described herein) may comprise nucleic acid barcode molecules as generally depicted in FIG. 12. In some embodiments, nucleic acid barcode molecules 1210 and 1212 are attached to support 1230 via a releasable linkage 1240 (e.g., comprising a labile bond) as described elsewhere herein. Nucleic acid barcode molecule 1210 may comprise adapter sequence 1211, barcode sequence 1212 and adapter sequence 1213. Nucleic acid barcode molecule 1220 may comprise adapter sequence 1221, barcode sequence 1212, and adapter sequence 1223, wherein adapter sequence 1223 comprises a different sequence than adapter sequence 1213. In some instances, adapter 1211 and adapter 1221 comprise the same sequence. In some instances, adapter 1211 and adapter 1221 comprise different sequences. Although support 1230 is shown comprising nucleic acid barcode molecules 1210 and 1220, any suitable number of barcode molecules comprising common barcode sequence 1212 are contemplated herein. For example, in some embodiments, support 1230 further comprises nucleic acid barcode molecule 1250. Nucleic acid barcode molecule 1250 may comprise adapter sequence 1251, barcode sequence 1212 and adapter sequence 1253, wherein adapter sequence 1253 comprises a different sequence than adapter sequence 1213 and 1223. In some instances, nucleic acid barcode molecules (e.g., 1210, 1220, 1250) comprise one or more additional functional sequences, such as a UMI or other sequences described herein. The nucleic acid barcode molecules 1210, 1220 or 1250 may interact with analytes as described elsewhere herein, for example, as depicted in FIGS. 13A-13C.

Referring to FIG. 13A, in an instance where cells are labelled with labeling agents, sequence 1323 may be complementary to an adapter sequence of a reporter oligonucleotide. Cells may be contacted with one or more reporter oligonucleotide 1310 conjugated labelling agents 1320 (e.g., polypeptide, antibody, or others described elsewhere herein). In some cases, the cells may be further processed prior to barcoding. For example, such processing steps may include one or more washing and/or cell sorting steps. In some instances, a cell that is bound to labelling agent 1320 which is conjugated to oligonucleotide 1310 and support 1330 (e.g., a bead, such as a gel bead) comprising nucleic acid barcode molecule 1390 is partitioned into a partition amongst a plurality of partitions (e.g., a droplet of a droplet emulsion or a well of a microwell array). In some instances, the partition comprises at most a single cell bound to labelling agent 1320. In some instances, reporter oligonucleotide 1310 conjugated to labelling agent 1320 (e.g., polypeptide, an antibody, pMHC molecule such as an MHC multimer, etc.) comprises a first adapter sequence 1311 (e.g., a primer sequence), a barcode sequence 1312 that identifies the labelling agent 1320 (e.g., the polypeptide, antibody, or peptide of a pMHC molecule or complex), and an adapter sequence 1313. Adapter sequence 1313 may be configured to hybridize to a complementary sequence, such as sequence 1323 present on a nucleic acid barcode molecule 1390. In some instances, oligonucleotide 1310 comprises one or more additional functional sequences, such as those described elsewhere herein.

Barcoded nucleic may be generated (e.g., via a nucleic acid reaction, such as nucleic acid extension or ligation) from the constructs described in FIGS. 13A-C. For example, sequence 1313 may then be hybridized to complementary sequence 1323 to generate (e.g., via a nucleic acid reaction, such as nucleic acid extension or ligation) a barcoded nucleic acid molecule comprising cell (e.g., partition specific) barcode sequence 1321 (or a reverse complement thereof) and reporter sequence 1312 (or a reverse complement thereof). Barcoded nucleic acid molecules can then be optionally processed as described elsewhere herein, e.g., to amplify the molecules and/or append sequencing platform specific sequences to the fragments. See, e.g., U.S. Pat. Pub. 2018/0105808, which is hereby entirely incorporated by reference for all purposes. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) may be performed. For example, the workflow may comprise a workflow as generally depicted in any of FIGS. 13A-13C, or a combination of workflows for an individual analyte, as described elsewhere herein. For example, by using a combination of the workflows as generally depicted in FIGS. 13A-13C, multiple analytes can be analyzed.

In some instances, analysis of an analyte (e.g. a nucleic acid, a polypeptide, a carbohydrate, a lipid, etc.) comprises a workflow as generally depicted in FIG. 13A. A nucleic acid barcode molecule 1390 may be co-partitioned with the one or more analytes. In some instances, nucleic acid barcode molecule 1390 is attached to a support 1330 (e.g., a bead, such as a gel bead), such as those described elsewhere herein. For example, nucleic acid barcode molecule 1390 may be attached to support 1330 via a releasable linkage 1340 (e.g., comprising a labile bond), such as those described elsewhere herein. Nucleic acid barcode molecule 1390 may comprise a barcode sequence 1321 and optionally comprise other additional sequences, for example, a UMI sequence 1322 (or other functional sequences described elsewhere herein). The nucleic acid barcode molecule 1390 may comprise a sequence 1323 that may be complementary to another nucleic acid sequence, such that it may hybridize to a particular sequence.

For example, sequence 1323 may comprise a poly-T sequence and may be used to hybridize to mRNA. Referring to FIG. 13C, in some embodiments, nucleic acid barcode molecule 1390 comprises sequence 1323 complementary to a sequence of RNA molecule 1360 from a cell. In some instances, sequence 1323 comprises a sequence specific for an RNA molecule. Sequence 1323 may comprise a known or targeted sequence or a random sequence. In some instances, a nucleic acid extension reaction may be performed, thereby generating a barcoded nucleic acid product comprising sequence 1323, the barcode sequence 1321, UMI sequence 1322, any other functional sequence, and a sequence corresponding to the RNA molecule 1360.

In another example, sequence 1323 may be complementary to an overhang sequence or an adapter sequence that has been appended to an analyte. For example, referring to FIG. 13B, in some embodiments, primer 1350 comprises a sequence complementary to a sequence of nucleic acid molecule 1360 (such as an RNA encoding for a BCR sequence) from an analyte carrier. In some instances, primer 1350 comprises one or more sequences 1351 that are not complementary to RNA molecule 1360. Sequence 1351 may be a functional sequence as described elsewhere herein, for example, an adapter sequence, a sequencing primer sequence, or a sequence the facilitates coupling to a flow cell of a sequencer. In some instances, primer 1350 comprises a poly-T sequence. In some instances, primer 1350 comprises a sequence complementary to a target sequence in an RNA molecule. In some instances, primer 1350 comprises a sequence complementary to a region of an immune molecule, such as the constant region of a TCR or BCR sequence. Primer 1350 is hybridized to nucleic acid molecule 1360 and complementary molecule 1370 is generated. For example, complementary molecule 1370 may be cDNA generated in a reverse transcription reaction. In some instances, an additional sequence may be appended to complementary molecule 1370. For example, the reverse transcriptase enzyme may be selected such that several non-templated bases 1380 (e.g., a poly-C sequence) are appended to the cDNA. In another example, a terminal transferase may also be used to append the additional sequence. Nucleic acid barcode molecule 1390 comprises a sequence 1324 complementary to the non-templated bases, and the reverse transcriptase performs a template switching reaction onto nucleic acid barcode molecule 1390 to generate a barcoded nucleic acid molecule comprising cell (e.g., partition specific) barcode sequence 1322 (or a reverse complement thereof) and a sequence of complementary molecule 1370 (or a portion thereof). In some instances, sequence 1323 comprises a sequence complementary to a region of an immune molecule, such as the constant region of a TCR or BCR sequence. Sequence 1323 is hybridized to nucleic acid molecule 1360 and a complementary molecule 1370 is generated. For example, complementary molecule 1370 may be generated in a reverse transcription reaction generating a barcoded nucleic acid molecule comprising cell (e.g., partition specific) barcode sequence 1322 (or a reverse complement thereof) and a sequence of complementary molecule 1370 (or a portion thereof). Additional methods and compositions suitable for barcoding cDNA generated from mRNA transcripts including those encoding V(D)J regions of an immune cell receptor and/or barcoding methods and composition including a template switch oligonucleotide are described in International Patent Application WO2018/075693, U.S. Patent Publication No. 2018/0105808, U.S. Pat. Publication No. 2015/0376609, filed Jun. 26, 2015, and U.S. Pat. Publication No. 2019/0367969, each of which applications is herein entirely incorporated by reference for all purposes.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Preparation of a MOF Nano-partition of a Reverse-Transcriptase Embedded in a Hydrogel Matrix

This example illustrates the preparation of porous metal-organic framework (MOF) nano-partition containing a reverse-transcriptase (RT) enzyme.

### Materials and Methods

Reverse transcriptase (RT) encapsulated in a zeolitic imidazolate framework (ZIF) type of metal organic framework (MOF) is synthesized according to the general method described in Shieh et. al., “”Imparting Functionality to Biocatalysts via Embedding Enzymes into Nanoporous Materials by a de Novo Approach: Size-Selective Sheltering of Catalase in Metal-Organic Framework Microcrystals,” JACS 137 (13): 4276-4279 (2015). Briefly, a stock solution of (i) 21 mg/mL imidazole-2-carboxaldehyde (ICA), (ii) 22 mg/mL polyvinyl pyrrolidone (avg. mw 40 k), and (iii) 0 or 7.5 mg/ml RT, is prepared, added dropwise into a flask with 124 mg/mL Zn(NO₃)₂ in DI water, and incubated at room temperature with stirring until precipitate is formed.

The resulting solid precipitate is isolated by centrifugation and washed with excess DI water, then vacuum-dried prior to resuspension in a buffered solution.

Dynamic light scattering is used to confirm that the MOF particle size is in the nm range. Additionally or alternatively, transmission electron microscopy is used to assess size and morphology of the MOF particles. Protein content of the MOF particles are assessed using standard assays, e.g., by BCA assay.

Activity of the ZIF-encapsulated RT is confirmed using standard RT activity assays. By way of example only, template switching efficiency of the encapsulated RT vs. non-encapsulated RT is assessed using capillary electrophoresis. By way of other example only, bulk RT reactions are carried out using Jurkat cells using encapsulated vs. non-encapsulated RT enzymes, in the presence or absence of proteinase K.

### Example 2: Preparation of a Discrete Droplet Containing a Fixed Biological Sample and an Un-Fixing Agent

This example illustrates preparation of a partition that is a discrete droplet (GEM) containing a fixed biological sample of PFA-fixed PBMCs, the un-fixing agent of compound (8), the protease, Proteinase K, a MOF nano-partition containing a reverse transcriptase, and assay reagents. A single-cell RNA sequence expression profiling experiment is then carried out using the droplets.

### Preparation of RT-containing Nano-Partition

A RT-containing nano-partition is prepared as described in Example 1.

### Preparation of Fixed Biological Sample

A fixed biological sample of fixed PBMCs is prepared as follows. PBMCs are fixed with 4% PFA for 24 h at 4° C. and quenched with 10% Fetal Bovine Serum (“FBS”) in PBS. The fixed biological sample can be stored at 4° C. or -20° C. for several days or more before being processed in a droplet-based assay (e.g., a single cell assay).

### Preparation of Un-Fixing Agent

The un-fixing agent of compound (8) is prepared using the following 2-step synthesis procedure.

Step 1: Diethyl (4-aminopyridin-3-yl)phosphonate. In step 1 the compound, diethyl (4-aminopyridin-3-yl)phosphonate is prepared according to the procedure described in Guilard, R. et al. Synthesis, 2008, 10, 1575-1579. Briefly, to a solution of 3-bromopyridine-4-amine (2.5 g, 14.5 mmol, 1 equiv) (CAS:13534-98-0, Sigma Aldrich) in ethanol (58 mL) is added diethyl phosphite (2.2 mL, 17.3 mmol, 1.2 equiv.) triethylamine (3 mL, 1.5 equiv), PPh₃(1.1 g, 4.3 mmol, 30 mol%) and Pd(OAc)₂ (0.39 g, 1.73 mmol, 12 mol%). The reaction mixture is purged with Argon for 5 min. After heating to reflux for 24 h, the reaction mixture is cooled to room T and concentrated in vacuo. The residue is purified by silica gel chromatography (MeOH/DCM) to yield the title compound (0.35 g, 11% yield). ¹H NMR (80 MHz, CDCl₃): δ = 1.15 (t, 6H, CH₃), 4.18-3.69 (m, 4H, CH₂), 5.99 (br-s, 2H, NH₂), 6.49 (d, 1H), 8.03-7.93 (m, 1H), 8.22 (d, 1H).

Step 2: 4-Aminopyridin-3-yl)phosphonic acid (compound (8). In step 2, the target compound, (4-Aminopyridin-3-yl)phosphonic acid (compound (8)) is prepared by acid hydrolysis of the precursor compound of step 1. Diethyl (4-aminopyridin-3-yl)phosphonate (0.35 g, 1.52 mmol, 1 equiv) is suspended in 6 N HCI (aq.) (8 mL). After refluxing for 12 h, the reaction mixture is concentrated in vacuo. The residue is washed with DCM, ether and concentrated in vacuo to afford the target compound (8) (247 mg, 93% yield). ¹H NMR (80 MHz, D₂O): δ = 6.85-6.55 (m, 1H), 8.05-7.94 (m, 1H), 8.40-8.26 (m, 1H).

Formulation of un-fixing agent solution comprising un-fixing agent and protease: The un-fixing agent of compound (8) is formulated in 30 mmol Tris buffer, pH 6.8 to a target concentration of 80 mM and the pH is adjusted to pH 6.5 using 2 M NaOH. A final formulation comprising 50 mM of compound (8), 0.2% SDS, 10 mU/mL of Proteinase K and 0.2 U/mL RNAse inhibitor in 30 mM Tris, pH 6.8, is generated. The final formulation is filtered through a 0.2 µm nylon syringe filter.

### Generation of Partitions Encapsulating Fixed PBMCs, Un-Fixing Solution, Barcoded Gel-Beads, and RT-Containing Nano-Partitions

The sample comprising fixed PBMCs is changed into a standard master mix used with the Chromium System (10X Genomics, Pleasanton, CA, USA) for partitioning samples together with the nano-partitions and barcoded gel beads in discrete droplets called GEMs (“Gel Beads in Emulsion”). The Chromium System is prepared with the un-fixing agent solution added as a separate reagent in generating the GEM containing the sample PBMC and the barcode gel bead. Alternatively, the un-fixing agent solution is added to the reservoir containing the suspension of barcoded gel-beads and introduced into the GEMs through the same inlet channel with the gel-beads.

Once generated, the GEMs are collected, and a heat incubation step is carried out. The heating step facilitates lysis and release of the cell contents, barcode oligonucleotides, and the reverse transcriptase (RT) catalyzed reaction that results in the cDNA synthesis reaction incorporating the barcodes in the 3′ synthons. In incorporating an un-fixing agent with the GEMs, the heat incubation step can be extended as necessary to allow for the un-fixing reaction catalyzed by compound (8) that removes the crosslinks from biomolecules released from the PBMC sample in the GEM.

While the foregoing disclosure of the present invention has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims.

Additional embodiments of the invention are set forth in the following claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

What is claimed is:

1. An assay method comprising:
(a) providing a partition containing a biological sample, a first enzyme, a second enzyme, and a nano-partition, wherein the first and second enzymes catalyze different reactions, and wherein the nano-partition separates the second enzyme from the biological sample and the first enzyme, thereby preventing the first and second enzymes from interacting;
(b) using the first enzyme to catalyze a reaction with the biological sample; and
(c) using the second enzyme to catalyze a reaction with the biological sample, or a component thereof;
optionally, wherein (b) and (c) occur simultaneously or wherein (c) occurs after (b).

2. (canceled)

3. (canceled)

4. The method of claim 1, wherein the reaction of the second enzyme with the biological sample generates analytes; optionally, wherein the method further comprises detecting the generated analytes.

5. (canceled)

6. The method of claim 1, wherein the reaction of the first enzyme with the biological sample generates a substrate for the reaction of the second enzyme; optionally wherein the generating comprises rendering the substrate accessible to the second enzyme.

7. The method of claim 1, wherein the first and second enzymes are incompatible; optionally, wherein the first enzyme degrades the second enzyme, the first enzyme reduces the activity of the second enzyme, and/or the first enzyme degrades a substrate or product of a reaction catalyzed by the second enzyme.

8. The method of claim 1, wherein the partition contains assay reagents.

9. The method of claim 1, wherein the nano-partition has a size of between about 3 nm and about 10,000 nm, between about 3 nm and about 1000 nm, or between about 3 nm and about 300 nm.

10. The method of claim 1, wherein the nano-partition comprises pores having an average diameter of less than about 5 nm, less than about 4 nm, less than about 3.5 nm, less than about 3 nm, or less than about 2 nm; or wherein the nano-partition comprises pores having an average diameter of between about 0.1 nm and about 10 nm, about 0.1 nm and about 5 nm, about 0.1 nm and about 3.5 nm, about 0.1 nm and about 2.5 nm, about 0.1 nm and about 2 nm, about 0.5 nm and about 10 nm, about 1 nm and about 8 nm, about 1.5 nm and about 6 nm, about 2 nm and about 5 nm, or about 2.3 nm and about 4 nm.

11. (canceled)

12. The method of claim 1, wherein the nano-partition comprises pores that allow the diffusion of nucleic acids; optionally, wherein the pores allow the diffusion of mRNA molecules.

13. The method of claim 1, wherein the nano-partition encapsulates the second enzyme.

14. The method of claim 1, wherein the nano-partition comprises a material selected from a metal organic framework, a hydrogel matrix, a dendrimersome, or a polymersome; optionally, wherein the nano-partition is a hydrogel matrix comprising cleavable crosslinks.

15. The method of claim 1, wherein the first enzyme is a protease; optionally, wherein the protease is selected from: alcalase, alkaline proteinase, ArcticZymes Proteinase, bacillopeptidase A, bacillopeptidase B, bioprase, colistinase, esperase, genenase, kazusase, maxatase, pepsin, Serratia peptidase, proteinase K, protease S, savinase, subtilisin A, subtilisin B, subtilisin BL, subtilisin E, subtilisin J, subtilisin S, subtilisin S41, thermoase, and trypsin, or a combination thereof.

16. The method of claim 1, wherein the second enzyme is a nucleic acid processing enzyme; optionally, wherein the nucleic acid processing enzyme is selected from: a reverse transcriptase, a polymerase, a terminal transferase, a transposase, a cas enzyme, a restriction enzyme, a USER enzyme, and/or a ligase.

17. The method of claim 1, wherein the partition is a discrete droplet; optionally, wherein the method further comprises emulsifying the droplet, thereby releasing its contents.

18. The method of claim 1, wherein:
(i) the partition further comprises a bead; optionally, wherein the bead comprises a plurality of nucleic acid barcode molecules; and/or
(ii) the partition further comprises one or more assay reagents, optionally wherein the one or more assay reagents comprise cDNA synthesis reagents; optionally, wherein the cDNA synthesis reagents comprise NTPs, primers, and template switch oligonucleotides.

19. (canceled)

20. The method of claim 1, wherein the biological sample is derived from a tissue sample, a biopsy sample, or a blood sample; or wherein the biological sample is or comprises a single cell, an organelle of a single cell, and/or a nucleus of a single cell.

21. (canceled)

22. The method of claim 1, wherein the biological sample is a fixed biological sample.

23. The method of claim 22, wherein the partition further comprises an un-fixing agent; optionally, wherein the un-fixing agent is a composition comprising a compound selected from: 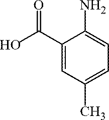
(1) 2-amino-5-methylbenzoic acid 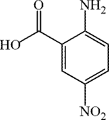 [SMILES: Nc1ccc([N+](=O)[O-])cc1C(=O)O]
(2) 2-amino-5-nitrobenzoic acid 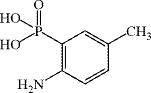
(3) (2-amino-5-methylphenyl)phosphonic acid 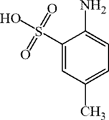
(4) 2-amino-5-methylbenzenesulfonic acid 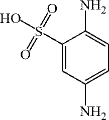 [SMILES: Nc1ccc(N)c(S(=O)(=O)O)c1]
(5) 2,5-diaminobenzenesulfonic acid 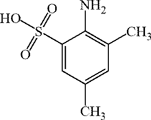
(6) 2-amino-3,5-dimethylbenzenesulfonic acid 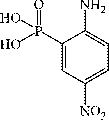 [SMILES: Nc1ccc([N+](=O)[O-])cc1P(=O)(O)O]
(7) (2-amino-5-nitrophenyl)phosphonic acid 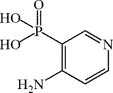 [SMILES: Nc1ccncc1P(=O)(O)O]
(8) (4-aminopyridin-3-yl)phosphonic acid 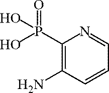 [SMILES: Nc1cccnc1P(=O)(O)O]
(9) (3-aminopyridin-2-yl)phosphonic acid 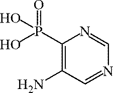 [SMILES: Nc1cncnc1P(=O)(O)O]
(10) (5-aminopyrimidin-4-yl)phosphonic acid 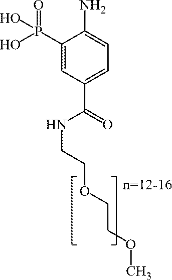
(11) (2-amino-5-{[2-(2-poly-ethoxy)ethyl]carbamoyl}phenyl)phosphonic acid
.

24. The method of claim 1, wherein the nano-partition is degradable and the method further comprises a step of providing a stimulus that degrades the nano-partition; optionally, wherein the nano-partition is degradable by a stimulus selected from heat, UV light, and a chemical reagent; optionally, wherein the chemical reagent is selected from DTT, DETA, EDA, TETA, hydrazine monohydrate, or a combination thereof.

25. (canceled)

26. The method of claim 24, wherein the stimulus that degrades the nano-partition also deactivates the first enzyme.

27. (canceled)

28. The method of claim 1, wherein the method further comprises a step of deactivating the first enzyme.

29-88. (canceled)
